(19) United States
(12) Patent Application Publication
(10) Pub. No.: US 2003/0040105 A1
(43) Pub. Date: Feb. 27, 2003

(54) Microfluidic micromixer

(76) Inventors: Larry A. Sklar, Albuquerque, NM (US); Tione Buranda, Albuquerque, NM (US); Bruce S. Edwards, Albuquerque, NM (US); Carlos M. Gallegos, Albuquerque, NM (US); W. Coyt Jackson, San Diego, CA (US); Frederick W. Kuckuck, Albuquerque, NM (US); Gabriel P. Lopez, Albuquerque, NM (US); Andrea A. Mammoli, Albuquerque, NM (US)

Correspondence Address: Ajay A. Jagtiani, Jagtiani + Guttag, Democracy Square Business Center, 10379-B Democracy Lane, Fairfax, VA, 22030, US

(21) Appl. No.: 10/021,243

(22) Filed: Dec. 19, 2001

Related U.S. Application Data
(63) Continuation-in-part of application No. 09/501,643, filed on Feb. 10, 2000.
(60) Provisional application No. 60/156,946, filed on Sep. 30, 1999.
(60) Provisional application No. 60/330,624, filed on Oct. 26, 2001.

(51) Int. Cl. C12M 1/34; B28C 5/06

(52) U.S. Cl. 435/287.2; 366/10

(57) ABSTRACT

A mixing apparatus is provided comprising: first driving means for driving a plurality of reagent samples from a plurality of respective source wells into a first fluid flow stream; second driving means for introducing a separation gas between each of the plurality of reagent samples in the first fluid flow stream; means for driving a second fluid flow stream comprising a plurality of particles; a junction device comprising: a first inlet port for receiving the first fluid flow stream; a second inlet port for receiving the second fluid flow stream; a reaction zone for forcing mixing between the first fluid flow stream and the second fluid flow stream to thereby form a reaction product stream; and an outlet port for allowing the reaction product stream to exit the junction device; a reaction zone where the plurality of reagent samples and the plurality of particles mix to form a plurality of reaction products, the reaction zone communicating with the outlet port; reaction product driving means for driving the reaction product stream through the reaction zone; and means for selectively analyzing the reaction product stream for the reaction products. A method for mixing materials is also provided comprising: driving a first fluid flow stream comprising a plurality of reagent samples separated by gas bubbles through a second inlet port of a junction device; driving a second fluid flow stream comprising particles through a first inlet port of the junction device; mixing the first fluid flow stream and the second fluid flow stream in a reaction zone in the junction device to form a reaction product stream; and driving the reaction product stream through an outlet port of the junction device.

### CROSS-REFERENCE TO RELATED APPLICATIONS

The present application makes reference to co-pending U.S. patent application Ser. No. 09/501,643 entitled “Flow Cytometry for High Throughput Screening” filed Feb. 10, 2000 that claims the priority of U.S. Provisional Patent Application No. 60/156,946, entitled “Flow Cytometry Real-Time Analysis of Molecular Interactions,” filed Nov. 9, 1999; and to co-pending U.S. Provisional Patent Application No. 60/330,624 entitled “In-Line Microfluidic Mixers for High Throughput Flow Cytometry,” filed on Oct. 26, 2001. The entire contents and disclosure of the above applications are hereby incorporated by reference.

### GOVERNMENT INTEREST STATEMENT

This invention is made with government support under Grant number GM60799 awarded by the National Institutes of Health. The government may have certain rights in this invention.

### BACKGROUND OF THE INVENTION

1. Field of the Invention

The present invention relates generally to micromixers.

2. Description of the Prior Art

Rapid advances have taken place in the biotechnology industry in the area of drug discovery, genomics, and proteomics. Currently, the devices being developed for use in these industries are microdevices where microliter or nanoliter volumes flow through micron dimension channels. The speed and ease with which mixing of multiple small volume samples can be achieved has a direct bearing on the success or failure of a number of these endeavors. However, the traditional integrated technologies for mixing small sample volumes is not suitable for most biological and pharmaceutical flow cytometry applications. Therefore, a mixing apparatus where samples and reagent samples may be mixed on-line along with the highly desirable features of the high throughput approach would be ideal for use by the biotech industry.

### SUMMARY OF THE INVENTION

It is therefore an object of the present invention to provide a microfluidic micromixer that may be used to mix small volume samples and reagent samples on-line.

It is a further object to provide a micromixer that will allow a mixing of small volumes flowing through micron dimension channels.

It is yet another object to provide a micromixer that will allow a mixing of multiple samples of microliter and nanoliter volumes.

It is yet another object to provide a micromixer where sample particles from one reservoir continuously can be mixed with different reagent samples efficiently without carryover.

According to a first broad aspect of the present invention, there is provided a microfluidic mixing apparatus comprising: first driving means for driving a plurality of reagent samples from a plurality of respective source wells into a first fluid flow stream; second driving means for introducing a separation gas between each of the plurality of reagent samples in the first fluid flow stream means for driving a second fluid flow stream comprising a plurality of particles; a junction device comprising: a first inlet port for receiving the first fluid flow stream; a second inlet port for receiving the second fluid flow stream; a reaction zone for forcing mixing between the first fluid flow stream and the second fluid flow stream to thereby form a reaction product stream; and a outlet port for allowing the reaction product stream to exit the junction device; a reaction zone where the plurality of reagent samples and the plurality of particles mix to form a plurality of reaction products, the reaction zone communicating with the outlet port; reaction product driving means for driving the reaction product stream through the reaction zone; and means for selectively analyzing the reaction product stream for the reaction products.

According to second broad aspect of the invention, there is provided a method for mixing materials comprising: driving a first fluid flow stream comprising a plurality of reagent samples separated by gas bubbles through a second inlet port of a junction device; driving a second fluid flow stream comprising particles through a first inlet port of a junction device; mixing the first fluid flow stream and the second fluid flow stream in a reaction zone in the junction device to form a reaction product stream; and driving the reaction product stream through an outlet port of the junction device.

Other objects and features of the present invention will be apparent from the following detailed description of the preferred embodiments.

### BRIEF DESCRIPTION OF THE DRAWINGS

The invention will be described in conjunction with the accompanying drawings, in which:

FIG. 1 illustrates in a schematic form a T mixing configuration constructed in accordance with a preferred embodiment of the invention;

FIG. 2 illustrates in a schematic form a Y mixing configuration constructed in accordance with a preferred embodiment of the invention;

FIG. 3 is a schematic partial view of reagent samples, particles and a reaction product flowing through tubing of a microfluidic mixing apparatus in accordance with an embodiment of the present invention;

FIG. 4 is a schematic partial view of reagent samples, particles and a reaction product flowing through tubing of a microfluidic mixing apparatus in accordance with an alternative embodiment of the present invention;

FIG. 5 is a schematic illustration of a first variation of a junction device of the present invention;

FIG. 6 is a schematic illustration of a second variation of a junction device of the present invention;

FIG. 7 is a schematic illustration of a third variation of a junction device of the present invention;

FIG. 8 is a schematic illustration of a fourth variation of a junction device of the present invention;

FIG. 9 is a schematic illustration of a fifth variation of a junction device of the present invention;

FIG. 10A is a dot plot analysis of red fluorescence of a mixing experiment conducted using a mixing system constructed in accordance with a preferred embodiment of the invention;

FIG. 10B is a dot plot analysis of green fluorescence of the mixing experiment of FIG. 10A;

FIG. 11 is a dot plot illustrating the transit of reagent samples through a microfluidic Y-junction mixing system of the present invention;

FIG. 12A is a schematic illustration of the process of biotin binding to streptavidin beads labeled with FITC-biotin;

FIG. 12B is a plot of data of mean channel fluorescence versus time for several concentrations of biotin of an experiment conducted using a mixing configuration of the present invention;

FIG. 13 is a schematic representation of an unquenching reaction performed using a microfluidic ‘Y’ mixing system of the present invention;

FIG. 14A is a dot plot of bead fluorescence versus time of an experiment conducted using the Y-shaped mixing system of FIG. 13;

FIG. 14B is a dot plot of marker beads versus time of an experiment conducted using the mixing system of FIG. 13;

FIG. 14C is an analysis showing plot of mean channel fluorescence versus time, overlaying the unquenching reaction and the marker beads of an experiment conducted using the mixing system of FIG. 13;

FIG. 15A is a dot plot of fluorescence unquenching versus time for manual mixing of the present invention;

FIG. 15B is a dot plot of fluorescence unquenching versus time for peristaltic delivery for mixing of the present invention;

FIG. 15C is a dot plot of fluorescence unquenching versus time using two syringes for mixing of the present invention;

FIG. 15D is a comparative plot of mean channel fluorescence quenching for manual mixing, a peristaltic pump and a syringe for mixing of the present invention;

FIG. 16A is an illustration of dot plot of fluorescence versus time for microfluidic mixing and continuous delivery for one run of an experiment using a microfluidic mixing system of the present invention;

FIG. 16B is an illustration of dot plot of fluorescence versus time for microfluidic mixing and continuous delivery for a second run of an experiment using a microfluidic mixing system of the present invention;

FIG. 16C is an illustration of dot plot of fluorescence versus time for microfluidic mixing and continuous delivery for a third run of an experiment using a microfluidic mixing system of the present invention;

FIG. 17A is a dot plot of fluorescence versus time for an experiment in which 6 reagent samples/minute were run through a microfluidic-junction of the present invention;

FIG. 17B is a dot plot of fluorescence versus time for an experiment in which 7 reagent samples/minute were run through a microfluidic-junction of the present invention; and

FIG. 17C is a dot plot of fluorescence versus time for an experiment in which 9 reagent samples/minute were run through a microfluidic-junction of the present invention.

### DETAILED DESCRIPTION OF THE PREFERRED EMBODIMENT

It is advantageous to define several terms before describing the invention. It should be appreciated that the following definitions are used throughout this application.

### Definitions

Where the definition of terms departs from the commonly used meaning of the term, applicant intends to utilize the definitions provided below, unless specifically indicated.

For the purposes of the present invention, the term “Reynolds number” refers to the function DUP/u used in fluid flow calculations to estimate whether flow through a pipe or conduit is streamline or turbulent in nature. D is the inside pipe diameter, U is the average velocity of flow, P is density, and U is the viscosity of the fluid. Reynolds number values much below 2100 correspond to laminar flow, while values above 3000 correspond to turbulent flow.

For the purposes of the present invention, the term “microfluidic” refers to the process where contents of two sample lines are mixed in a mixer. The bubbles separate discrete sample units. The pulsatile action in the fluid forces the discrete samples to mix with the continuously supplied material.

For the purposes of the present invention, the term “driven cavity” refers to the process where contents of two sample lines are mixed in a mixer, using the bubbles associated with the discrete sample units to mix the discrete sample units with the continuously drawn material. The bubbles force the fluid in the discrete samples to mix with the continuously supplied material. Thus, the driving force behind mixing is the bubbles. Preferably, the cavity is “driven” when the mixing of fluids is achieved by turbulence caused by the bubbles.

For the purposes of the present invention, the term “pulsatile fluid motion” refers to the motion that is created in the fluid as a result of being driven by a peristaltic pump.

For the purposes of the present invention, the term “pulsatile fluid mixing” refers to the process where contents of two sample lines are mixed in a mixer, using the pulsatile fluid motion associated with the discrete or discontinuous sample units to mix the discontinuous sample units with the continuously drawn material. The pulsatile fluid motion forces the fluid in the discontinuous samples to mix with the continuously supplied material also propelled by pulsatile fluid motion. Thus, the driving force behind mixing is the pulsatile fluid motion.

For the purposes of the present invention, the term “diameter” refers to the maximum cross sectional inner dimension of a device through which a fluid flows such as a tube, channel, pore, etc.

For the purposes of the present invention, the term “micromixer” refers to a mixer where microliter volumes are mixed.

For the purposes of the present invention, the term “nanomixer” refers to a mixer where nanoliter volumes are mixed.

For the purposes of the present invention, the term “microchannels” refers to channels having a diameter of ˜0.01 inch=0.0254 cm.

For the purposes of the present invention, the term “discontinuous sample” refers to discrete sample units preceded and followed by air bubbles.

For the purposes of the present invention, the term “particles” refers to any particles such as beads or cells that may be detected using a flow cytometry apparatus, whether in solution or suspension, etc. The particles to be analyzed in a sample may be tagged, such as with a fluorescent tag. The particles to be analyzed may also be bound to a bead, a cell, a receptor, or other useful protein or polypeptide, or may just be present as free particles, such as particles found naturally in a cell lysate, purified particles from a cell lysate, particles from a tissue culture, etc. When the particles to be analyzed are biomaterials, drugs may be added to the reagent samples to cause a reaction or response in the particles with which the reagent samples are mixed.

For the purposes of the present invention, the term “biomaterial” refers to any organic material obtained from an organism, either living or dead. The term “biomaterial” also refers to any synthesized biological material such as synthesized oligonucleotides, synthesized polypeptides, etc. The synthesized biological material may be a synthetic version of a naturally occurring biological material or a non-naturally occurring biological made from portions of naturally occurring biological materials, such as a fusion protein, or two biological materials that have been bound together, such as an oligonucleotide, such as DNA or RNA, bound to a peptide, either covalently or non-covalently, that the oligonucleotide does not normally bind to in nature.

For the purposes of the present invention, the term “source well” refers to any well on a well plate, whether or not the source well contains a reagent sample. For the purposes of the present invention, the term “reagent sample source well” refers to a source well containing a reagent sample.

For the purposes of the present invention, the term “reagent sample” refers to a fluid solution or suspension containing solids, such as beads to which a reagent has been bound, to be mixed with “particles” using a method and/or apparatus of the present invention. The reagent sample may include chemicals, either organic or inorganic, used to produce a reaction with the particles to be analyzed.

For the purposes of the present invention, the term “adjacent samples” refers to two samples in a fluid flow stream that are separated from each other by a separation gas, such as an air bubble. For the purposes of the present invention, the term “immediately adjacent samples” refers to adjacent samples that are only separated from each other by a separation gas. For the purposes of the present invention, “buffer fluid separated adjacent samples” refers to adjacent samples that are separated from each other by two separation gas bubbles and a buffer fluid, with the buffer fluid being located between the two separation gas bubbles.

For the purposes of the present invention, the term “separation gas” refers to any gas such as air, an inert gas etc. that can be used to form a gas bubble between adjacent samples or between a sample and a buffer fluid.

For the purposes of the present invention, the term “buffer fluid” refers to a fluid that is substantially free of the particles to be detected by the apparatus and method of the present invention.

For the purposes of the present invention, the term “drug” refers to any type of substance that is commonly considered a drug. For the purposes of the present invention, a drug may be a substance that acts on the central nervous system of an individual, e.g. a narcotic, hallucinogen, barbiturate, or a psychotropic drug. For the purposes of the present invention, a drug may also be a substance that kills or inactivates disease-causing infectious organisms. In addition, for the purposes of the present invention, a drug may be a substance that affects the activity of a specific cell, bodily organ or function. A drug may be an organic or inorganic chemical, a biomaterial, etc. The term drug also refers to any molecule that is being tested as a potential precursor of a drug.

For the purposes of the present invention, the term “plurality” refers to two or more of anything, such as a plurality of samples.

For the purposes of the present invention, the term “homogenous” refers to a plurality of identical samples. The term “homogenous” also refers to a plurality of samples that are indistinguishable with respect to a particular property being measured by an apparatus or a method of the present invention.

For the purposes of the present invention, the term “heterogeneous” refers to a plurality of samples in a fluid flow stream in which there are at least two different types of reagent samples in the fluid flow stream. One way a heterogeneous plurality of samples in a fluid flow stream of the present invention may be obtained is by intaking different reagent samples from different source wells in a well plate.

For the purposes of the present invention, the term “fluid flow stream” refers to a stream of fluid that is contained in a fluid flow path such as a tube, a channel, etc. A fluid flow stream may include one or more bubbles of a separation gas and/or one or more portions of a buffer fluid.

For the purposes of the present invention, the term “fluid flow path” refers to device such as a tube, channel, etc. through which a fluid flow stream flows. A fluid flow path may be composed of several separate devices, such as a number of connected or joined pieces of tubing or a single piece of tubing, alone or in combination with channels or other different devices.

For the purposes of the present invention, the term “high speed multi-sample tube” refers to any tube that may be used with a peristaltic pump that has compression characteristics that allow a peristaltic pump to move samples separated by a separation gas through the tube at a speed of at least 6 samples per minute without causing adjacent samples to mix with each other. The polyvinylchloride (PVC) tube may have an inner diameter of about 0.001 to 0.03 inches. An example of such a tube is a PVC tube having an inner diameter of about 0.005 to 0.02 inches and a wall thickness of about 0.01 to 0.03 inches. A particularly preferred tube is a PVC tube having an inner diameter of about 0.01 inches and a wall thickness of about 0.02 inches.

For the purposes of the present invention the term “aqueous buffer with physiological saline” refers to 150 mM NaCl.

For the purposes of the present invention the term “reaction zone” refers to a space where a reaction may occur within the space. The reaction that occurs in the reaction zone may be between any reagent sample and/or any particles. For example, the reaction zone may be a cavity.

### Description

Over the last few years, there has been a lot of interest in developing methods of mixing solutes in microchannels. The evolving interest in the miniaturization of bench-scale biochemical or chemical processes into sub-microliter or nanoliter systems was the motivation that spurred research in the direction of mixing solutes in microchannels. The cross-sectional dimensions associated with these miniaturized systems are of the order of micrometers to millimeters. As diffusion is predominant in these length scales, focus in the design of mixers has been to utilize mixing by molecular diffusion.

A variety of mixers have been designed for continuous-flow systems, where two liquids streams are made to flow through a channel such that the liquids are mixed during their residence time in the channel. For a given velocity of the fluid, the residence time of the liquid is increased, by increasing the length of the channel so as to ensure complete mixing. In some designs, the mixer channel is branched into multiple narrower channels so as to ensure mixing in a shorter residence time.

As opposed to continuous-flow systems, complete mixing in systems where one component is present continuously and the other component is presented in discrete units without any carryover is generally not possible using current technologies.

The present invention provides a flow approach that allows rapid delivery of multiple samples consisting of multiple cells or particles and their detection. The approach of the present invention uses a high throughput microfluidic mixing apparatus. A preferred high throughput apparatus of the present invention includes an auto sampler to pick reagent samples from a multi-well plate, bubbles to separate reagent samples and a pump to deliver the reagent samples to a junction device where they are mixed with a stream of particles.

The present invention provides a simple, general, continuous high throughput method for mixing and delivery of sub microliter volumes in laminar flow at low Reynolds number. Normally, at low Reynolds number, fluids that are introduced through a T or Y-junction travel in laminar flow and do not mix. The present invention provides a way of mixing, using a microfluidic-junction with a general approach consistent with flow-through detection systems sensitive to submicroliter volumes. In a preferred embodiment, a microfluidic-junction of the present invention employs two streams of fluid traveling through a T or Y-junction, but with sequential samples separated by bubbles. Mixing occurs through pulsatile action.

This micromixing approach of the present invention is compatible with commercial autosamplers, flow cytometry and other detection schemes that require mixing of components that have been introduced into laminar flow. The present invention provides a way of achieving on-line mixing in conjunction with the high throughput approach. In a preferred embodiment of the present invention, individual reagents present in wells are drawn by one sample line. Bubbles are inserted between the compounds to separate the compounds into discrete units. Another separate sample line draws a solution containing cells or particles from a reservoir in a continuous manner. The contents of the two fluid flow lines are mixed in a T or Y-junction, using the bubbles associated with the discrete compound units to mix the discrete compound units into units with continuously drawn sample. The pulsatile motion forces the fluid in the discrete samples to mix with the continuously supplied sample.

A factor that affects mixing of fluids is the Reynolds number. Normally at a low Reynolds number, typically less than 1, the fluid in a T-junction flows in a linear fashion. Only convective mixing as a result of diffusion by Brownian motion takes place at low Reynolds number. Mixing by diffusion alone takes too long to provide an efficient means of mixing. At a Reynolds number above 2300 there is turbulent flow, and fluid mixes very well. In intermediate ranges, mixing reflects an intermediate trend. The Reynolds number has a dependence on the diameter of tubing through which fluid flows. With wider diameter tubing, fluid flow is slower and the Reynolds number is lower. The smaller the diameter of the tubing through which a fluid flows, generally the faster the flow rate, and the higher the Reynolds number.

A preferred embodiment of the present invention allows mixing to occur of small volumes flowing through micron dimension channels or tubing, even at a low Reynolds number by providing a “microfluidic pulsatile action”. The dimensions of the tubing used to make the mixer of the present invention and introduction of bubbles between samples to ensure complete mixing play important roles in present invention. The approach can be generalized from flow cytometry to microfluidic channels of internal diameter ˜0.01 inch similar to the tubing used to deliver samples to a flow cytometer.

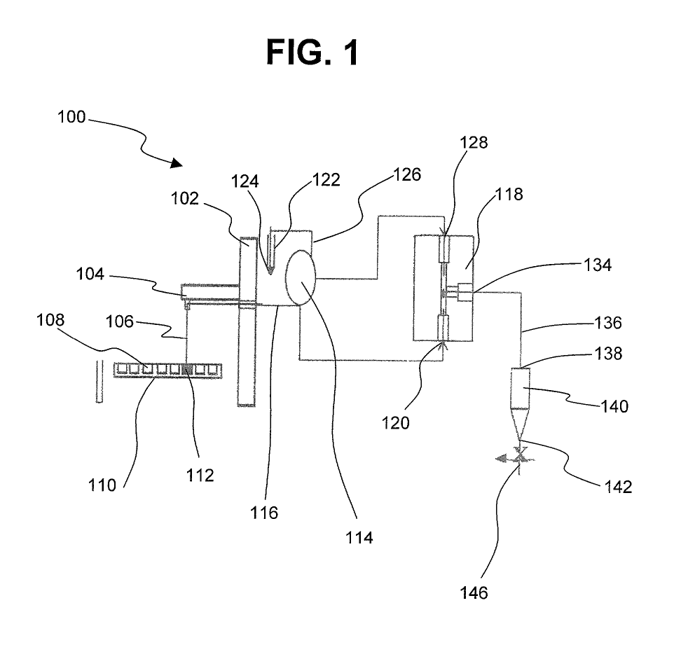

FIG. 1 illustrates a preferred microfluidic mixing system 100 of the present invention. Microfluidic mixing system 100 includes an autosampler 102 having an adjustable port 104 on which is mounted a hollow probe 106. As port 104 moves back and forth, left and right in FIG. 1, and side to side, into and out of the plane of FIG. 1, a probe 106 is lowered into individual source wells 108 of a well plate 110 to obtain a reagent sample 112 that has been tagged with a fluorescent tag. Once a reagent sample 112 is picked up by probe 106, a peristaltic pump 114 forces reagent sample 112 through a tube 116 that extends from autosampler 102 through peristaltic pump 114 and connects to T-junction 118 at an inlet port 120. A reservoir 122 contains particles 124 that have been tagged with a different fluorescent tag. An alternative embodiment of the present invention may use particles that have been differentially tagged. A tube 126 picks up particles 124 from reservoir 122. Peristaltic pump 114 forces particles 124 through tube 126 that extends from bead sample (not shown) through peristaltic pump 114 and connects to T-junction 118 at an inlet port 128. T-junction 118 consists of top or right inlet port 128 where particles 124 from reservoir 122 enter T-junction 118. Mixing of particles 124 and reagent sample 112 takes place in T-junction 118 to form a reaction product (not shown). The reaction product is driven through an outlet port 134 of T-junction 118 and into an outlet reaction product tube 136. Outlet reaction product tube 136 carries the samples mixed in T-junction 118 through an interrogation point 138 of a detection system 140. Outlet reaction product tube 136 includes a reaction zone 142 in which any reaction between particles 124 and reagent sample 112 takes place. Detection system 140 includes a flow cytometer (not shown) consisting of a flow cell 146 and a laser interrogation device (not shown). Laser interrogation device (not shown) examines individual samples flowing from flow cell 146 at interrogation point 138.

In between intaking reagent samples 112 from each of source wells 108, probe 106 is allowed to intake air (or other gas), thereby forming an air bubble (not shown) between each adjacent sample (not shown). Individual reagents present in source wells 108, are drawn by tube 116. Bubbles are inserted between reagent samples 112 to separate the compounds. In addition, some of source wells 108 may include a buffer solution that may be periodically intaken between reagent samples.

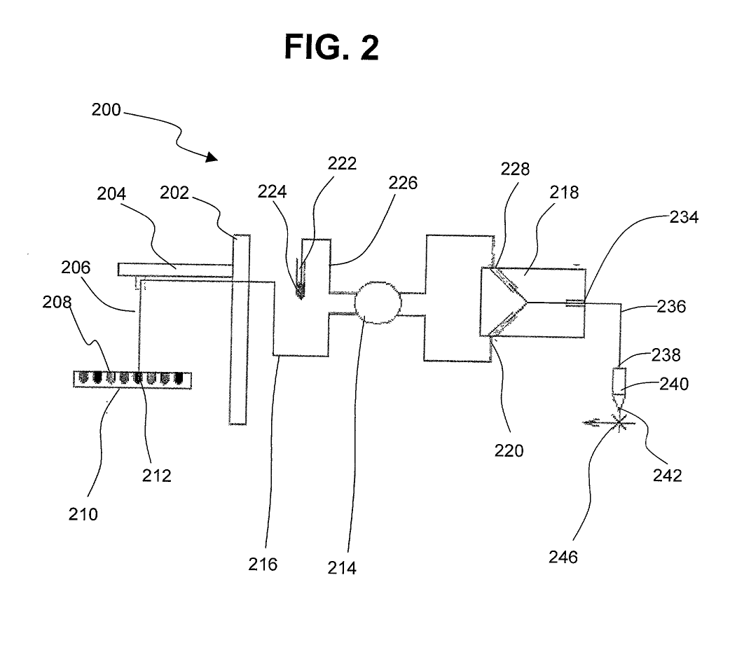

FIG. 2 illustrates a preferred microfluidic mixing system 200 of the present invention. Microfluidic mixing system 200 includes an autosampler 202 having an adjustable port 204 on which is mounted a hollow probe 206. As port 204 moves back and forth, left and right in FIG. 2, and side to side, into and out of the plane of FIG. 2, probe 206 is lowered into individual source wells 208 of a well plate 210 to obtain a reagent sample 212 that has been tagged with a fluorescent tag. Once a reagent sample 212 is picked up by probe 206, a peristaltic pump 214 forces reagent sample 212 through a tube 216 that extends from autosampler 202 through peristaltic pump 214 and connects to Y-junction 218 at an inlet port 220. A reservoir 222 contains particles 224 that have been tagged with a different fluorescent tag. An alternative embodiment of the present invention may use particles that have been differentially tagged. A tube 226 picks up particles 224 from reservoir 222. Peristaltic pump 214 forces particles 224 through tube 226 that extends from bead sample (not shown) through peristaltic pump 214 and connects to Y-junction 218 at an inlet port 228. Y-junction 218 consists of top or right inlet port 228 where particles 224 from reservoir 222 enter Y-junction 218. Mixing of particles 224 and reagent sample 212 takes place in Y-junction 218 to form a reaction product (not shown). The reaction product is driven through an outlet port 234 of Y-junction 218 and into an outlet reaction product tube 236. Outlet reaction product tube 236 carries the samples mixed in Y-junction 218 through an interrogation point 238 of a detection system 240. Outlet tube 236 includes a reaction zone 242 in which any reaction between particles 224 and reagent sample 212 take place. Detection system 240 includes a flow cytometer (not shown) consisting of a flow cell (not shown) and a laser interrogation device (not shown). Laser interrogation device (not shown) examines individual samples flowing from flow cell 246 at interrogation point 238.

In between intaking reagent samples 212 from each of source wells 208, probe 206 is allowed to intake air (or other gas), thereby forming an air bubble (not shown) between each adjacent sample (not shown). Individual reagents present in source wells 208 are drawn by tube 216. Bubbles are inserted between reagent samples 212 to separate the compounds. In addition, some of source wells 208 may include a buffer solution that may be periodically intaken between reagent samples.

Microfluidic mixing systems employing Y-junctions of the type shown in FIG. 2 are preferred for applications where carryover of successive samples has to be limited. Although the Y-junction device of FIG. 2 is shown having the inlets and outlets spaced evenly at 1200, other angular configurations may be employed for the Y-junction of the present invention.

Preferably the particles in the particle solutions of the embodiments of the invention shown in FIGS. 1 and 2 are bound to beads.

Although the reaction zones shown in FIGS. 1 and 2 are only a portion of the outlet tube, the reaction zone may include almost the entire outlet tube prior to the detection device.

Although a single peristaltic pump is shown in FIGS. 1 and 2 to drive reagent samples and particles through their respective tubes, in an alternative embodiment of the present invention, separate pumps may be used to drive the reagent samples and the particles.

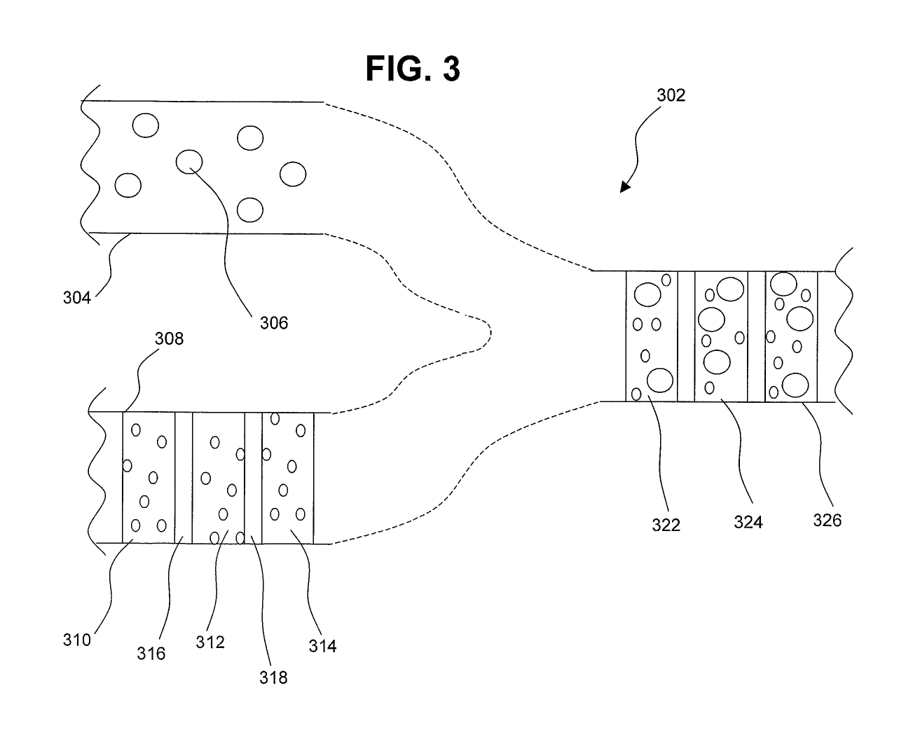

FIG. 3 illustrates a fluid flow system 302 of the present invention that may be used in a microfluidic mixing system of the present invention, such as the microfluidic mixing system of FIG. 1 or the microfluidic mixing system of FIG. 2. Fluid flow system 302 includes a tube 304 through which a particle solution 306 flows and a tube 308 through which reagent samples 310, 312 and 314 flow to mix together with particle solution 306 in a junction device. Immediately adjacent reagent samples 310 and 312 are separated from each other by air bubble 316, and immediately adjacent reagent samples 312 and 314 are separated from each other by air bubble 318.

Flowing from the junction device is a series of reaction product samples 322, 324, 326 that contain a mixture of particle solution 306 with reagent samples 310, 312 and 314 respectively. When reaction product samples 322, 324, 326 pass through an interrogation point (not shown), the particles in reaction product samples 322, 324 and 326 are sensed by a flow cytometer (not shown) due to the fluorescent tag(s) on the particles. In contrast, when air bubbles 316 and 318 pass through the interrogation point, no particles are sensed. Therefore, a graph of the data points of fluorescence sensed versus time for a series of samples analyzed using the flow cytometer of the present invention will form distinct groups, each aligned with the time that a sample containing particles passes through the laser interrogation point. In order to detect the presence of each of two or more different types of samples, in a heterogeneous plurality of samples, each of the two or more different types of samples may be tagged with different fluorescent tags, different amounts of a single tag or some combination of different tags and different amounts of a single tag. In such a case, the groupings of data points will vary vertically on a fluorescence versus time graph, depending on which type of sample is being sensed. As with the case of sensing a single type of sample, each sensed sample will exhibit a group of data points aligned with the time that the sample passes through the laser interrogation point.

FIG. 4 illustrates another fluid flow system 402 of the present invention that may be used in a microfluidic mixing system of the present invention, such as the microfluidic microfluidic mixing system of FIG. 1 or the microfluidic mixing system of FIG. 2. Fluid flow system 402 includes a tube 404 through which a particle solution 406 flows and a tube 408 through which reagent samples 410, 412 and 414 flow to mix together with particle solution 406 in a junction device. Adjacent reagent samples 410 and 412 are separated by air bubbles 416 and 417 and buffer solution 418, and adjacent reagent samples 412 and 414 are separated by air bubbles 420 and 421 and buffer solution 422.

Flowing from the junction device is a series of reaction product samples 432, 434, 436 that contain a mixture of particle solution 406 with reagent samples 410, 412 and 414 respectively. When reaction product samples 432, 434, 436 pass through an interrogation point (not shown), the particles in reaction product samples 432, 434 and 436 are sensed by a flow cytometer (not shown) due to the fluorescent tag on the particles. In contrast, when air bubbles 416, 417, 420 and 421 and buffer solution 418 and 422 pass through the interrogation point, no particles are sensed. Therefore, a graph of the data points of fluorescence sensed versus time for a series of samples analyzed using the flow cytometer of the present invention will form distinct groups, each aligned with the time that a sample containing particles passes through the laser interrogation point. In order to detect the presence of each of two or more different types of samples, in a heterogeneous plurality of samples, each of the two or more different types of samples may be tagged with different fluorescent tags, different amounts of a single tag or some combination of different tags and different amount of a single tag. In such a case, the groupings of data points will vary vertically on fluorescence versus time graph, depending on which type of sample is being sensed. As with the case of sensing a single type of sample, each sensed sample will exhibit a group of data points aligned with the time that the sample passes through the laser interrogation point.

In order to provide buffer solution separated adjacent reagent samples as shown in FIG. 4, some of the source wells on the well plate of a microfluidic mixing system of the present invention, such as the microfluidic mixing systems of FIGS. 1 and 2, may contain a buffer solution to allow for the formation of buffer fluid separated adjacent reagent samples in a tube through which samples pass. When this is the case, after each reagent sample is picked up by the probe, the probe intakes air, then is lowered into a source well containing buffer solution, then the probe intakes air again, and then the probe intakes a second reagent sample. This sequence may then be repeated for samples which the probe subsequently intakes.

Alternatively, buffer fluid separated adjacent reagent samples may be formed by providing a reservoir of buffer fluid in or attached to the autosampler to inject buffer fluid into the tube for the fluid flow stream. In this case, after each sample is picked up by the probe, the probe intakes air, then buffer fluid is injected into the tube for the fluid flow stream, then the probe intakes air again, and then the probe intakes a second sample. This sequence may then be repeated for subsequent samples to be separated by a buffer fluid.

The present invention is compatible with relatively inexpensive commercial well plates for use with autosamplers from 60 well plates to 72 well plates to 96 well plates to 384 well plates to at least as many as 1536 well plates. The source wells of the present invention may be all filled with samples and/or buffer fluids, or some may be left empty. When there are a plurality of different types of samples in the source wells of a well plate, the sample types may be arranged in the order in which they are taken up by the probe, or the sample types may be arranged in any other convenient arrangement. For example, all of the source wells in one row of source wells may contain one sample type and all of the source wells of a second row may contain a second sample type.

The source wells may be made any conventional shape used for source wells in a well plate for an autosampler. Preferably, when small amounts of sample are used in each source well, the source wells are conical in shape, as illustrated in FIG. 1, to allow even the smallest amounts of sample to be withdrawn by the probe or to allow the particles to concentrate in the bottom of the well. The use of a well plate with conical source wells reduces the problems associated with the settling of particles to the bottom of the well prior to being intaken by the probe. An alternative means to circumvent particle settling would be to sample from wells in an inverted plate given appropriate well dimensions that will permit sample retention in the well, e.g. by capillary forces or surface tension, when the plate is in this position.

The autosampler of the present invention may be any conventional autosampler suitable for intaking samples from a well plate. A preferred type of autosampler is the Gilson 215 liquid manager.

The use of automation in plate delivery and retrieval for the autosampler may allow automation of the overall screening process.

One preferred probe for the present invention is a 0.01 inch inner diameter, 1/15 inch outer diameter stainless steel needle compatible with HPLC ferrule fittings. A Gilson interface module for bidirectional communication between an MS DOS computer and a probe manipulating port and peristaltic pump. Software, such as QuickSip™, designed using commercial languages, such as Microsoft Visual C++, may be used to control the speed and distance of probe motions in all 3 dimensions, the sensing of probe contact with liquid in a source well to assure reproducible sample volumes, and the speed of the peristaltic pump. A computer or other known device may be used to control the autosampler to regulate sample size and bubble size by varying the time that the probe is in a source well or above a source well. Also, various sample handlers and sampler handling systems that may be useful in the apparatus and method of the present invention are well known in the art. One example of an integrated handler and programmable station is the Beckman 1000 Laboratory Workstation TM robotic which may be adapted for use in the apparatus or method of the present invention.

In order to reduce carryover, the probe may have a conical tip. Use of silicone or other hydrophobic agent to coat the tip of the sampling probe may also be helpful to minimize sample carryover. Alternatively, the entire probe may be made of a hydrophobic material to reduce carryover. Suitable hydrophobic materials for used in the coating or for making the entire hydrophobic probe include: Teflon® (poly(tetrafluoroethylene) (PTFE)), Kynar® (polyvinylidene fluoride), Tefzel® (ethylene-tetrafluoroethylene copolymer), tetrafluoroethylene-perfluoroalkyl vinyl ether copolymer resin (PFA), a tetrafluoroethylene-hexafluoropropylene copolymer (EFP), polyether ether ketone (PEEK), etc.

In order to reduce sample carryover, a jet of gas, such as air, may be sprayed on the tip of the autosampler probe. The source of the jet of gas may be mounted either on the autosampler or near the autosampler. Another way to reduce sample carryover is to use a rinsing device that may be attached to the autosampler or be otherwise mounted on or near the flow cytometry apparatus of the present invention to rinse the autosampler probe between intakes of sample and/or buffer solution. The rinsing fluid may be water, a mild detergent, or a solvent, such as a solvent in which each of the particles in one or more of the samples is dissolved. When the particles are merely suspended in a suspension fluid, the rinsing fluid may be the same as the suspension fluid. The use of an autosampler with a sensing probe tip may improve the efficiency of sample uptake and performance by reducing carryover and ensuring reproducible sample volumes.

Various conventional peristaltic pumps may be used with the flow cytometry apparatus of the present invention. A preferred peristaltic pump is Gilson Minipuls 3 for one embodiment of the present invention. Preferably, a peristaltic pump of the present invention is operated in a manner that reduces pulsatile flow, thereby improving the sample characteristics in the flow cytometer. For example, a tubing length greater than 20 inches between pump and flow cytometer may be used or a linear peristaltic pump such as the Digicare LP5100 may be used to improve the sample characteristics. However, for another embodiment of the present invention where mixing is dependent on pulsatile fluid motion the peristaltic pump is operated in a manner that generates pulsatile flow.

Various types of tubing may be used for the fluid flow path of the present invention, as long as the tubing may function as high speed multi-sample tubing. When thin walled PVC (polyvinyl chloride) tubing is used as the tubing for the present invention, carryover between samples is substantially reduced compared to conventional peristaltic tubing. Preferably, the fluid flow path of the present invention is a single length of tubing without—unnecessary junctions. Such a single length of tubing reduces the breakup of bubbles and improves the performance in sample separation. A preferred type of high speed multi-sample tubing for use with the present invention is about 0.001 to about 0.03 inch inner diameter PVC tubing having a wall thickness of about 0.01 to about 0.03 inches. A particularly preferred tubing is 0.01 inch inner diameter PVC tubing having a wall thickness of 0.02 inch.

Various types of flow cytometers may be used with the microfluidic mixing system of the present invention. Preferred types of flow cytometers are described in U.S. Pat. Nos. 5,895,764; 5,824,269; 5,395,588; 4,661,913; the entire contents and disclosures of which are hereby incorporated by reference. In a flow cytometer, samples may be sorted on a particle by particle basis using known methods. The flow cytometer may use software gating by light scatter to reduce the “noise” in the flow cytometer introduced by the periodic appearance of bubbles. The use of real-time software in conjunction with flow cytometer controlling software may allow the samples from a given source well to be re-checked during sampling and data analysis to prove that “hits” from neighboring source wells do not arise from cross-contamination.

On-line data analysis may be used in a flow cytometer to compare data between well plates and facilitate overall utility of the data in conjunction with automation. Operation of a flow cytometer at higher pressure generally increases the sample flow rate and may, in some circumstances, yield a higher throughput. Also, operation of the flow cytometer with increased time resolution in data software may allow resolution of samples at higher throughput rates.

A number of factors affect the performance of the fluids in a mixer. Dimensions of the tubing through which the fluid is flowing affects the mixing. Diameter of the tubing that allows efficient microfluidic mixing is in the range of 0.05 cm to 0.0125 cm.

Length of the mixer itself plays an important role in microfluidic mixing. As samples travel through the mixer it gives time for cells to interact with the stimulus, biomaterial, or drug. So the size of the mixer plays a key role in ensuring the online reactions go to completion.

Various types of buffer solutions may be used in the present invention. Although one type of preferred buffer solution are aqueous buffers with physiological saline, other types of buffers may be used in the present invention depending on the particles or reagent samples used in the present invention. Mammalian cells are sensitive to the buffer salinity, while beads may not be.

The sample volumes that can be mixed with the microfluidic mixing system of the present invention are submicroliter in volume and samples can be mixed at rates up to at least 100/samples per minute. However, with the geometry of the current invention, carry over between samples occurs within the Y that may be reduced if the mixing system is flushed with several volumes of buffer. Throughputs for screening of at least 20 samples per minute may be achieved. Taken together, the high throughput approach and the microfluidic mixing systems of the present invention serve to integrate autosamplers with submicroliter detection volumes for analysis in flow cytometry or in microfluidic channels.

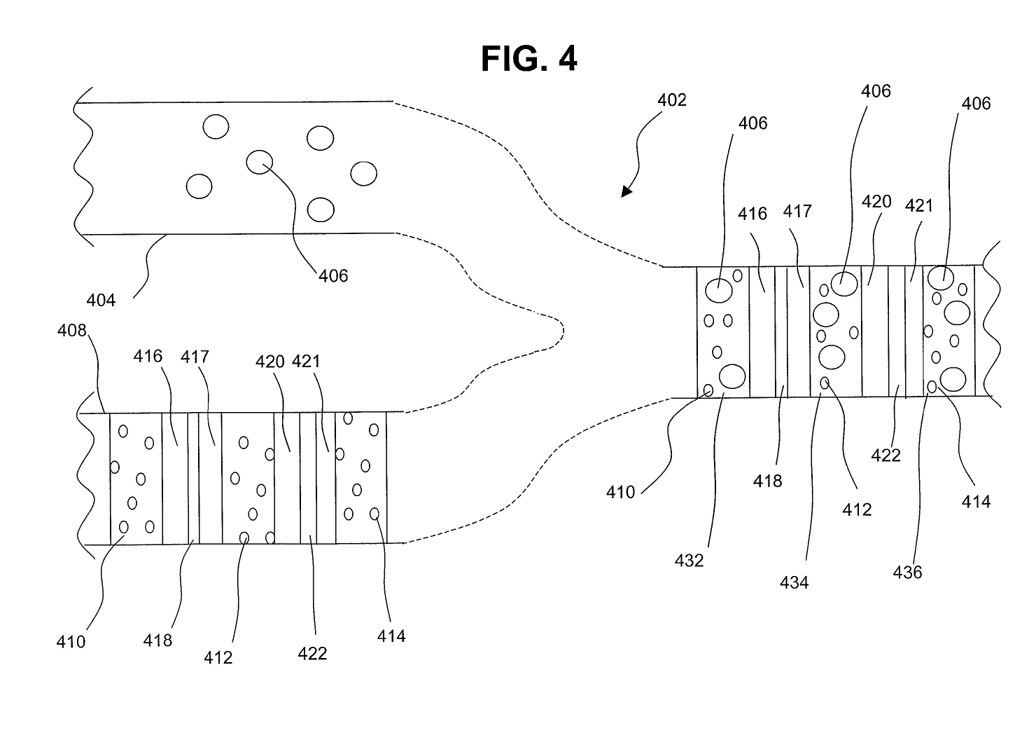

FIG. 5 is a schematic illustration of a first variation of a Y-junction device 500 of the present invention. Device 500 is composed of first inlet port 502, a second inlet port 504 and an outlet port 506. The internal diameter ID1 of ports 502, 504 and 506 is identical. An inlet tube 508 is fitted over inlet port 502. An inlet tube 510 is fitted over inlet port 504. An outlet tube 512 is fitted over outlet port 506. The connections of tubes 508, 510 and 512 with ports 502, 504, and 506, respectively, result in a distortion of the internal diameter of tubing 508, 510 and 512 at the points 514, 516 and 518. The difference AID in the internal diameter ID2 of tubing 508, 510 and 512 and internal ID1 results in dead volume spaces at points 514, 516 and 518. The angle between any two pairs of ports of Y-junction device 500 is 120°.

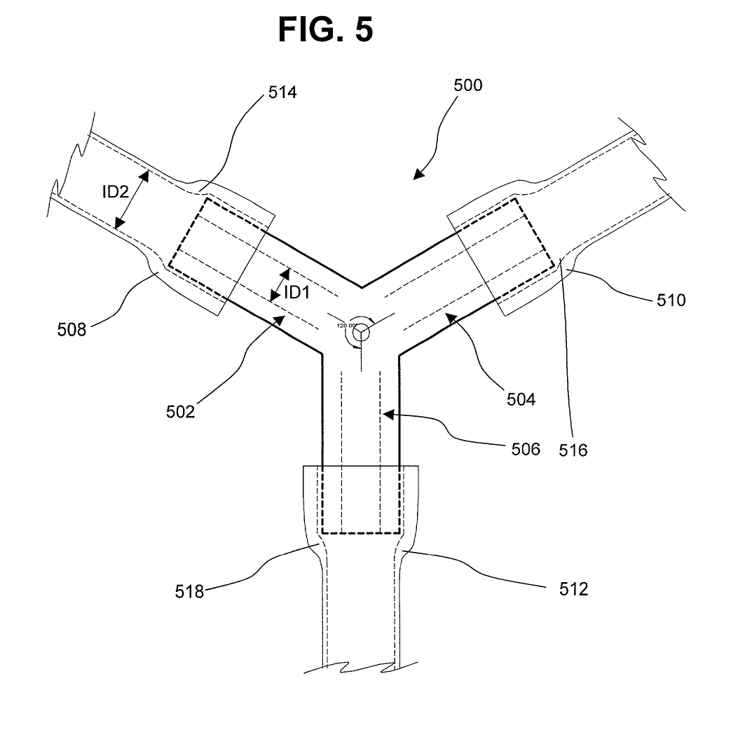

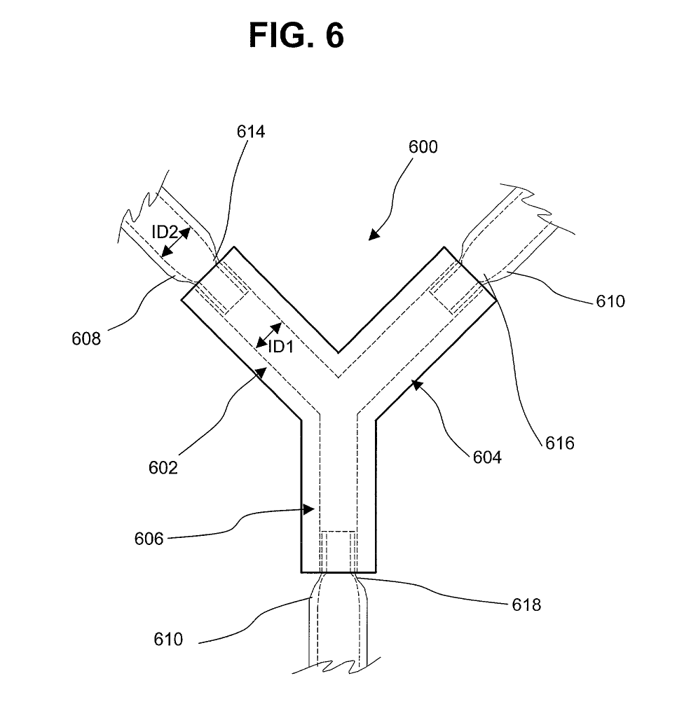

FIG. 6 is a schematic illustration of a first variation of a Y-junction device 600 of the present invention. Device 600 is composed of first inlet port 602, a second inlet port 604 and an outlet port 606. The internal diameter ID1 of ports 602, 604 and 606 is identical. An inlet tube 608 is fitted into inlet port 602. An inlet tube 610 is fitted into inlet port 604. An outlet tube 612 is fitted into outlet port 606. The connections of tubes 608, 610 and 612 with ports 602, 604, and 606, respectively, result in a distortion of the internal diameter of tubing 608, 610 and 612 at the points 614, 616 and 618. The difference AID in the internal diameter ID2 of tubing 608, 610, and 612 and internal ID1 results in dead volume spaces at points 614, 616 and 618.

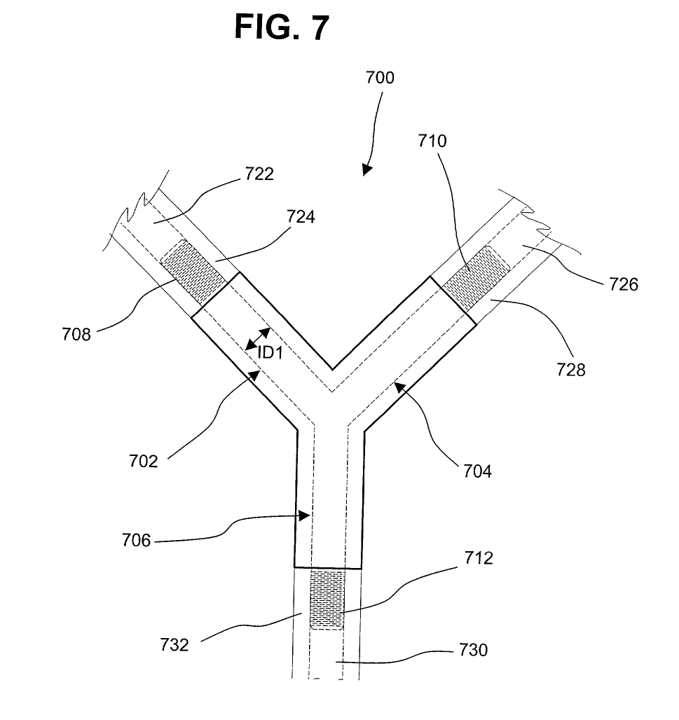

It may be important for some other embodiments of the present invention to be composed of a Y-junction device where the internal diameter of the entire mixing configuration is uniform. FIG. 7 depicts a Y-junction device 700 of the present invention where the internal diameter ID of the entire mixing configuration is uniform. Y-junction device 700 is composed of an inlet port 702, an inlet port 704 and an outlet port 706. The internal diameter ID of three ports 702, 704 and 706 are identical. Ports 702, 704 and 706 include male screw ends 708, 710 and 712, respectively. An inlet tube 722 includes a female screw end 724 that screws onto male screw end 708 to mate inlet tube 722 with inlet port 702. An inlet tube 726 includes a female screw end 728 that screws onto male screw end 710 to mate inlet tube 726 with inlet port 704. An outlet tube 730 includes a female screw end 732 that screws onto male screw end 712 to mate outlet tube 730 with outlet port 706. The arrangement described in FIG. 7 results in the internal diameter ID of ports 702, 704, 706 and tubes 722, 726 and 730 being uniform with no dead volume spaces.

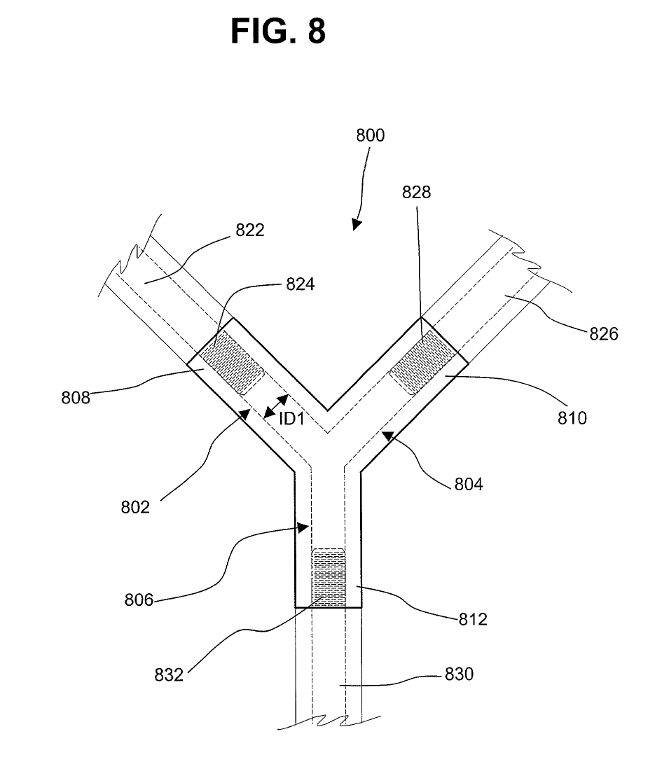

FIG. 8 depicts another Y-junction device 800 of the present invention where the internal diameter ID of the entire mixing configuration is uniform. Y-junction device 800 is composed of an inlet port 802, an inlet port 804 and an outlet port 806. The internal diameter ID of three ports 802, 804 and 806 are identical. Ports 802, 804 and 806 include female screw ends 808, 810 and 812, respectively. An inlet tube 822 includes a male screw end 824 that screws into female screw end 808 to mate inlet tube 822 with inlet port 802. An inlet tube 826 includes a male screw end 828 that screws into female screw end 810 to mate inlet tube 826 with inlet port 804. An outlet tube 830 includes a male screw end 832 that screws into female screw end 812 to mate outlet tube 830 with outlet port 806. The arrangement described in FIG. 8 results in the internal diameter ID of ports 802, 804, 806 and tubes 822, 826 and 830 being uniform with no dead volume spaces.

Although FIGS. 7 and 8 illustrate embodiments of the present invention in which the two inlet tubes/ports and the one outlet tube/port have identical internal diameter, in some applications one or more of the tube/port connections may have an internal diameter different from the other tube/port connections.

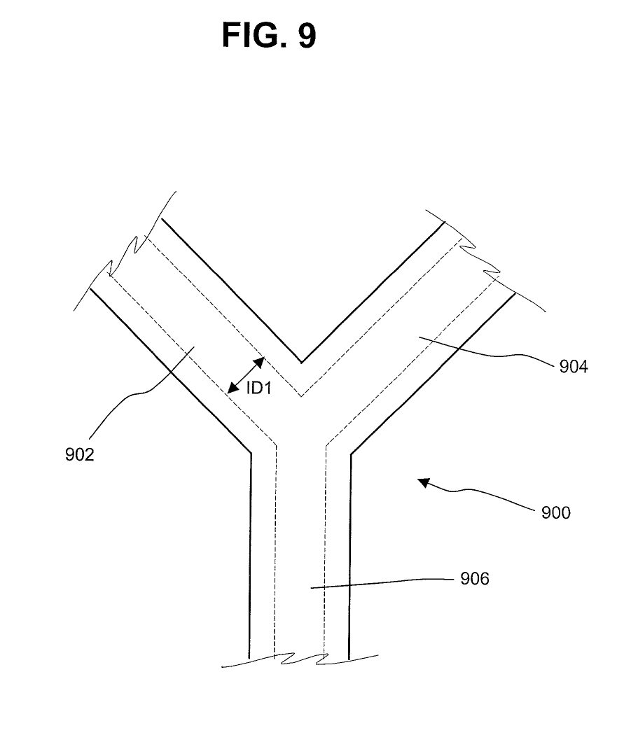

FIG. 9 illustrates another embodiment of the microfluidic Y junction mixing device 900 where the entire mixing device has a unibody construction. Mixing device 900 comprises two inlet ports/tubes 902 and 904 and an outlet port 906, each having an internal diameter ID. The entire device 900 has a unibody construction, so there are no screws or overlapping connections that connect the inlet and outlet tubes to the Y-junction.

Although FIG. 9 illustrates an embodiment of the present invention in which the three ports/tubes have the same internal diameter, in some applications one or more of the tube/port connections may have an internal diameter different from the other tube/port connections.

Although the Y-junction devices illustrated in FIGS. 5, 6, 7, 8 and 9 have ports that are spaced, for example at 120°, the angles between the various ports may be varied for different applications.

The present invention will now be described by way of example.

### EXAMPLE 1

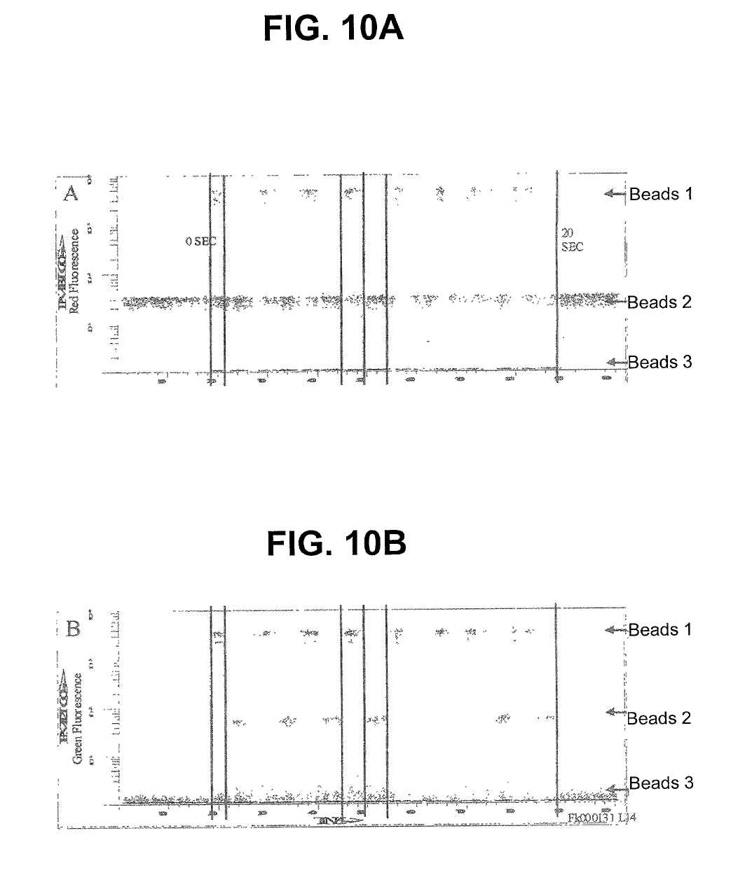

FIGS. 10A and 10B are a dot plot analysis of a mixing experiment conducted using a mixing configuration constructed in accordance with a preferred embodiment of the present invention. The experiment was conducted using a T-junction mixer. Three populations of beads were used in this experiment. Beads 1 have bright red and bright green fluorescence associated with them. Beads 2 have intermediate red and dim green fluorescence associated with them. While beads 3 have dim red and intermediate green fluorescence associated with them.

Beads 1 and 3 were separated from each other by bubbles and were being delivered to the T-junction by the same sample line intermittently from alternating wells on a microplate. Beads 2 were delivered continuously to the T-junction by a different sample line. The results of this mixing experiment were analyzed using red fluorescence in FIG. 10A as well as green fluorescence in FIG. 10B.

FIG. 10A shows that beads 2 are being detected continuously as intermediate red fluorescence. Beads 1 are detectable intermittently as bright red fluorescence. FIG. 10B shows that beads 2 are being detected continuously as dim green fluorescence. Beads 1 and beads 3 are being detected alternately as bright green and intermediate green fluorescent spots respectively.

### EXAMPLE 2

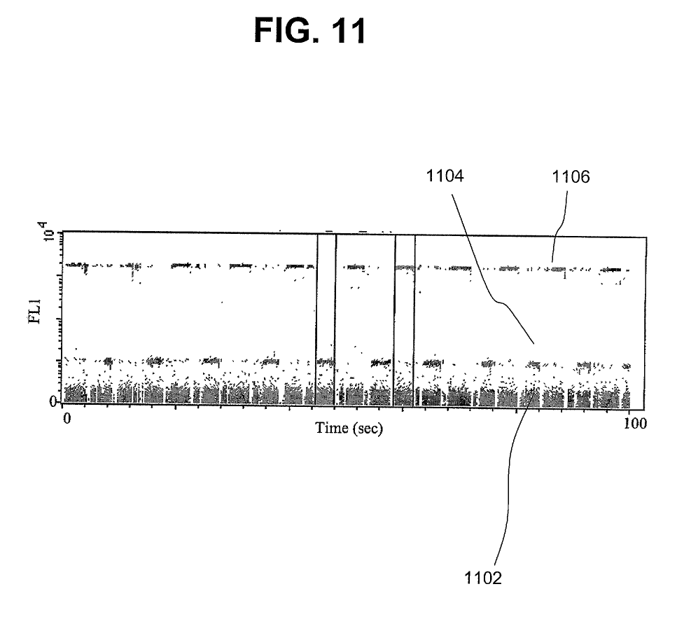

A Y shaped mixer was used in a series of experiments. The dimensions of junction inlets were: inner diameter of 0.0175 cm and dimension of the junction outlet was inner diameter of 0.0254 cm. In the present case, a solution of particles flowing continuously is brought together in a Y with reagent samples from wells, which were separated by bubbles. In the effluent stream, the particles and reagent samples were mixed and the reagent samples from individual wells were resolved. The flow cytometer graph of FIG. 11, shows the continuous appearance of the beads 1102 from the reservoir and the alternating appearance of samples 1104 and 1106 from the wells of the microtiter plate. While the alternating samples 1104 and 1106 were separated by bubbles, it is also clear that bubbles, breaks in data stream, occur in a somewhat irregular fashion. Even though the continuous flow and alternating flow occur simultaneously it is not clear that mixing is occurring. Normally, at a low Reynolds number, the particles entering from both of the Y ports would continue in laminar flow, without a uniform mixing.

### EXAMPLE 3

A flow cytometer detection system was used to carry out several experiments in high throughput screening using a microfluidic mixing system of the present invention. Experiments were performed on a Becton Dickinson FacScan (San Jose, Calif.) equipped with a 488 Argon ion laser. Samples from a 96-well plate were delivered to the flow cytometer using a Gilson 215 autosampler (Middleton, Wis.). A Gilson interface module allowed for bi-directional communication between an MS DOS computer, a robotic probe port and peristaltic pump. The probe was a 0.305 meter long (508 μm OD×254 μm ID) stainless steel tube (Small Parts Inc., Miami Lakes, Fla.). The probe was then attached to 177 μm (0.0075 in) ID flexible PVC tubing (Spectra Hardware Inc., Westmoreland City, Pa.) 1.5 meters in length and run through a peristaltic pump and attached to one leg of a ‘Y’-connector, 0.005 inch to 0.02 ID (Small Parts Inc.). The other leg of the Y-connector was coupled to similar tubing delivering a continuous stream of streptavidin coated beads. The flow cytometer was connected via 1.5 meter tubing (254 μm, 0.01 in ID) attached to the third leg of the ‘Y’. All legs of the ‘Y’ fitting have an inner diameter of 254 um with a centralized triangular dead volume of 0.14 μL. The arrangement of the apparatus minimized sample disruption between the narrow ID delivery tubing and the flow cytometer intake tube, ID 0.016 inch.

In-house software, QuickSip™, written in Microsoft Visual C⁺⁺, was used to control the speed and distance of probe motions in three dimensions. The speed of the peristaltic pump was manually controlled. Sample size and bubble size, were regulated by varying the time the probe was in a well or above a well intaking air. In a typical experiment, the peristaltic pump ran at 15 RPM. Sample plugs were removed from wells at sampling times of 400 ms per well. Sample-separating bubbles were generated during the time the probe was in transit between sample intakes (˜300 ms). The size of the sample plug was also regulated by the initial volume of sample in a given well. The biotin plugs were made smaller by keeping the initial volume of the biotin wells at 100 μL compared to the rinse wells, which contained 300 μL of buffer. For the sampling sequence comprising the repetitive delivery of a 3.85×10⁻⁶ M biotin plug and 9 rinses, the resultant stream of plugs consists of an estimated 0.6 μL of biotin and 0.9 μL of neat TRIS buffer. The biotin and buffer plugs subsequently combined with the continuous stream of fluorescein biotin-bearing beads at the Y-junction. To track the onset of each biotin plug after mixing at the Y-junction, Flow Check beads were included in each biotin sample well. The 96 well plate was periodically agitated to minimize settling of bead suspensions.

Data Analysis

Cell Quest software (Becton-Dickinson) was used to acquire time-resolved event clusters generated by rapid multi-well sampling. Event clusters representing the bead/biotin interactions were immediately identified based on changes in fluorescence intensity and automatically analyzed via software algorithms. The algorithms calculate mean and median fluorescence intensity as well as event number and standard deviation of each event cluster. More detailed analysis such as washout sequences and sample carryover identification was done in Microsoft Excel via the off-line analysis of the data files using flow cytometry list-mode data files stored in FCS 2.0 format. It is worth noting that data acquisition occurs continuously which includes air bubbles and fluid plugs simultaneously. The air bubbles between each fluid plug are denoted by gaps in event clusters and signal discontinuity.

Cell-Mimetic Assay System

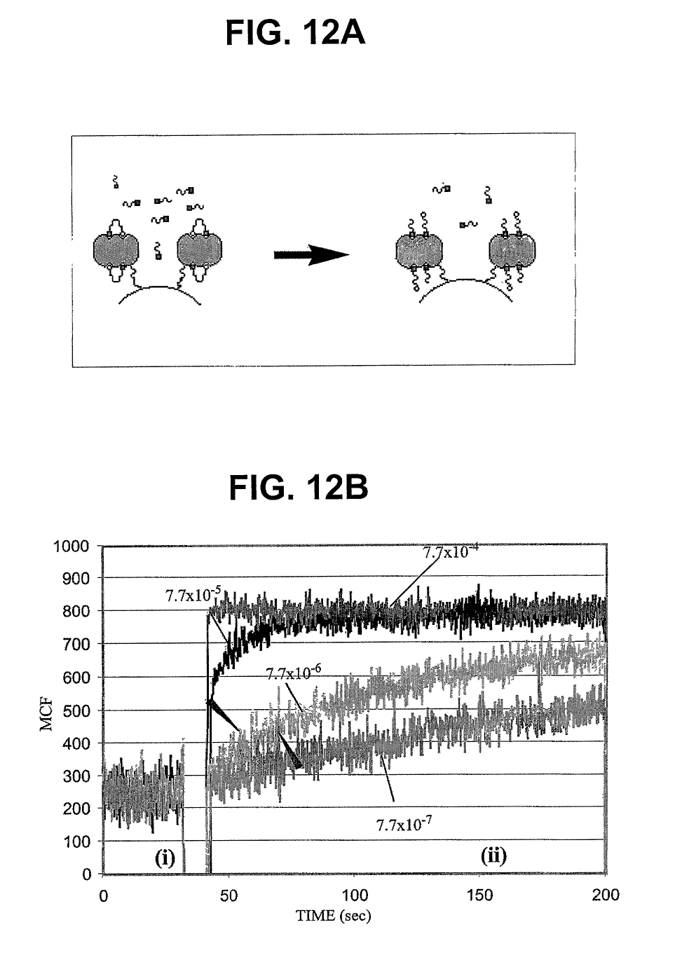

In order to verify mixing, a particulate system to mimic cell responses was developed. Optimally, the binding of a small molecule to a cell surface or particle would cause an increased signal over a desired time-span in the absence of extraneous fluorescence, such as would occur in cells in which receptor agonists elevate intracellular calcium in the presence of an intracellular calcium dye. FIG. 12A illustrates the detection of biotin binding kinetics to streptavidin beads labeled with FITC-biotin. Data are plotted as mean channel fluorescence (MCF) versus time for several concentrations of biotin. The binding of fluorescein biotin to streptavidin-coated beads shown in FIG. 12A is characterized by the quenching of fluorescence of particles bound relative to free fluorescein biotin. The quenching is relieved when free biotin is added. The base line fluorescence indicated by the constant signal on the right signifies the “ostrich quenched” fluorescein biotin beads. Typically, this type of quenching, “ostrich quenching,” occurs when the fluorescein moiety associates with the streptavidin-binding pocket adjacent to the biotin-moiety bearing site FIG. 12A. This interaction for fluorescein biotin is very weak K_{d}≈0.1 and readily obstructed by native biotin. Addition of native biotin allows the recovery of the original intensity under diffusion-limited kinetics as shown in FIG. 12B. The subsequent increase in fluorescence signal on right is caused by homogeneously mixing native biotin with the microspheres causing the unquenching of FITC-biotin in the streptavidin receptor pocket on the bead surface. For the beads used here an eight-fold increase in fluorescence intensity after mixing with excess native biotin was typical. The characteristic streptavidin coated bead used here was previously determined to bear on the order of 20 million biotin receptor sites. FIG. 12B shows time resolved fluorescence increase of fluorescein biotin beads (1.1×10⁵ beads bearing ≈1×10⁶ ostrich quenched fluorescein biotins/bead) after mixing various concentrations of native biotin and analyzing by flow cytometry. By varying the native biotin from 7.7×10⁻⁷ M to 7.7×10⁻⁴ M concentrations, the kinetics of the reaction are regulated. The arrows signify the responses expected for microfluidic mixing at the specified concentration and mixing time. At 7.7×10⁻⁷ M concentration of biotin a sample plug of 2 feet length mixes in 12 seconds by the present microfluidic mixing system while at a concentration of 7.7×10⁻⁶ M biotin a sample plug of 5 ft length mixes in 27 seconds by the microfluidic system.

Sample Transit

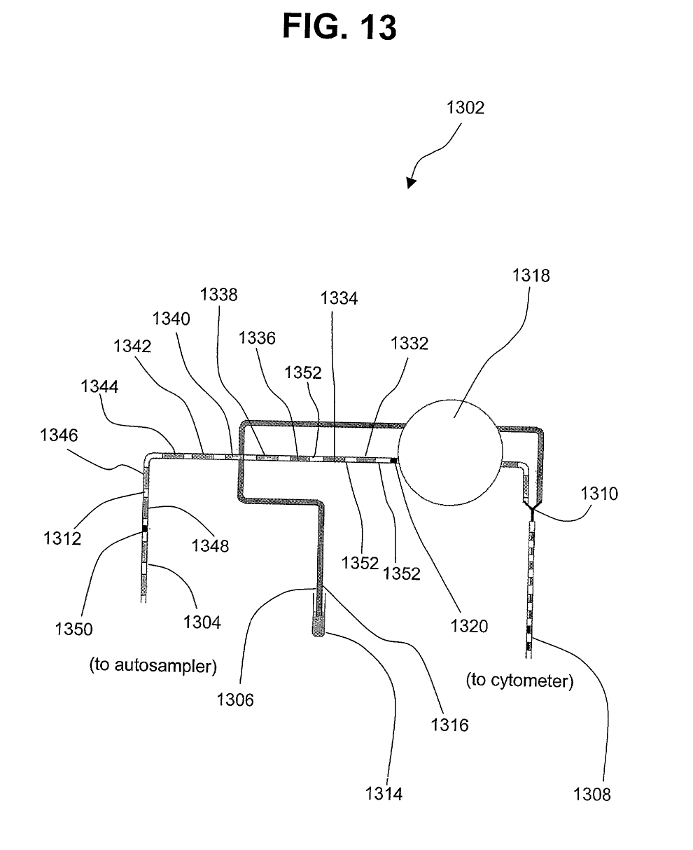

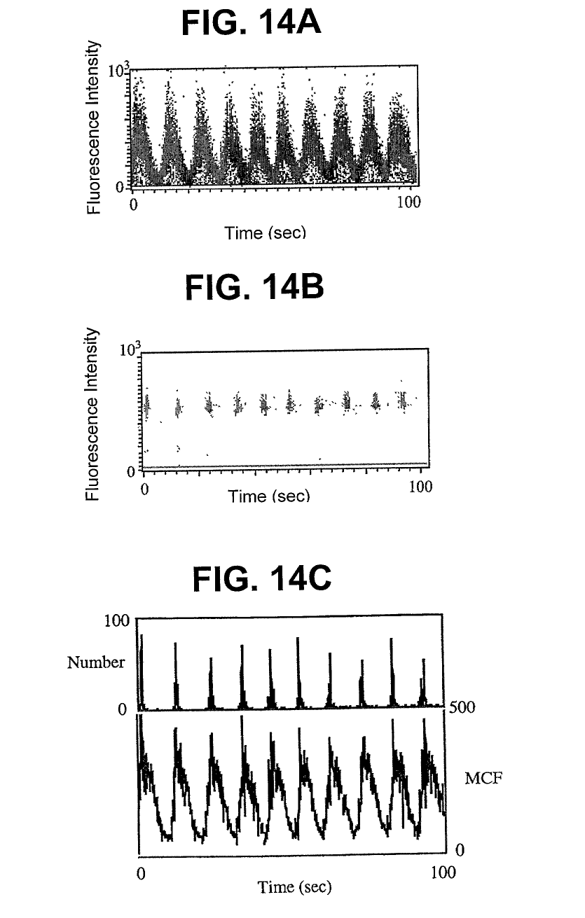

The rapid sampling of biotin and associated rinse plugs and the mixing downstream with the ostrich-quenched beads is illustrated by the schematic representation of a delivery sequence for unquenching reaction in FIG. 13 and performance of the microfluidic-junction shown in FIGS. 14A, 14B and 14C.

FIG. 13 illustrates a microfluidic mixing system 1302 of the present invention that was used to carry out an unquenching reaction. Microfluidic mixing system 1302 includes a reagent sample inlet tube 1304, a particle inlet tube 1306, and an outlet tube 1308 that are each connected to a Y-junction device 1310. Reagent sample inlet tube 1304 was also connected to an autosampler (not shown) for intaking a discontinuous stream 1312 of reagent samples. Particle inlet tube 1306 is connected to a reservoir 1314 of streptavidin coated FITC-biotin labeled microspheres that are intaken as a continuous particle stream 1316 into particle inlet tube 1306 by the action of peristaltic pump 1318. Peristaltic pump 1318 which was set at 15 RPM was used to drive reagent samples 1312 through reagent sample inlet tube 1304 and to drive microspheres 1316 through particle inlet tube 1306. A probe (not shown) of the autosampler intook in sequence from respective wells of a well plate (not shown) one sample 1320 of 7.7×10⁻⁶ M biotin containing “marker” beads followed by nine buffer rinse units 1332, 1334, 1336, 1338, 1340, 1342, 1344, 1346 and 1348, and another biotin bead sample 1350. In between intakes of sample and buffer, air was intaken by the probe to form air plugs 1352. The intake time was 0.4 sec and the transit time between wells while taking in air about 0.3 seconds. The discontinuous reagent sample stream 1312 was then mixed with the continuous particle stream 1316 in Y-junction device 1310. Microfluidic mixing occurred when discontinuous reagent sample stream 1312 and continuous particle stream 1316 convectively combined in outlet tube 1308 having an internal diameter of 0.01 inch (254 μm).

The outlet tube had a length ranging from 2-5 ft in the experiments. The total transit time for the reagent sample and nine buffer samples to flow into the Y-junction device to mix with the continuous flow of beads ranged from 12-27 seconds. The performance of the above-described system is characterized in FIGS. 14A, 14B, and 14C.

FIG. 14A depicts a dot plot of bead fluorescence versus time. Ten cycles of sample delivery are shown. Each cycle is characterized by the unquenching reaction indicating the mixing of biotin with marker beads with FITC-biotin labeled microspheres indicated by the peak followed by washout of the biotin delivered from blank sample wells. The addition of biotin is indicated by the onset and the washout by the disappearance of fluorescence of the beads FIG. 14A.

FIG. 14B is depicting a dot plot of marker beads versus time. The timing of the addition of biotin is indicated by the appearance of marker beads also contained in the biotin well FIG. 14B.

FIG. 14C is an analysis of data files showing mean fluorescence versus time, overlaying the unquenching reaction and the marker beads. FIG. 14C shows the overlay and temporal match between the distribution of marker beads and the onset of fluorescence intensity increase. The vertical discontinuities in intensity shown in FIG. 14C correspond to the passage of bubbles past the detector during which no particles are detected. Parallel experiments were performed with a T of comparable dimensions. In this case, the marker beads distribute irregularly over time (data not shown).

It was apparent that mixing occurred in the situation where bubbles separate samples of particles in the presence of the low molecular weight, readily diffusible, biotin reagent of MW ˜100. As expected, the degree of unquenching increased if a longer reaction time was allowed in a longer length of tubing (data not shown). While it is known that if the particles and biotin were in a laminar flow at a low Reynolds number established upon exiting the Y, mixing could occur by diffusion. In fact, similar unquenching occurs if bubbles are used to separate short samples or if a continuous stream of biotin and beads originating from the branches exit the Y in a more or less undisturbed laminar flow (data not shown).

The attenuation of fluid handling systems to the micron range involves a regime where small Reynolds numbers govern the delivery of fluid samples. As fluid transport systems get progressively smaller, viscous forces will tend to dominate over inertial forces, as turbulence becomes nonexistent. This leaves diffusion as the principal method of mixing of reagents. The typical diffusion coefficient for biomolecules is on the order of ≦10⁻⁷ cm² s⁻¹; thus mixing by diffusion is slow. For example, it would take ten days for a large protein to diffuse a distance of 1 cm by diffusion alone. Mixing of reagents has thus been a barrier to sample delivery in miniaturized systems.

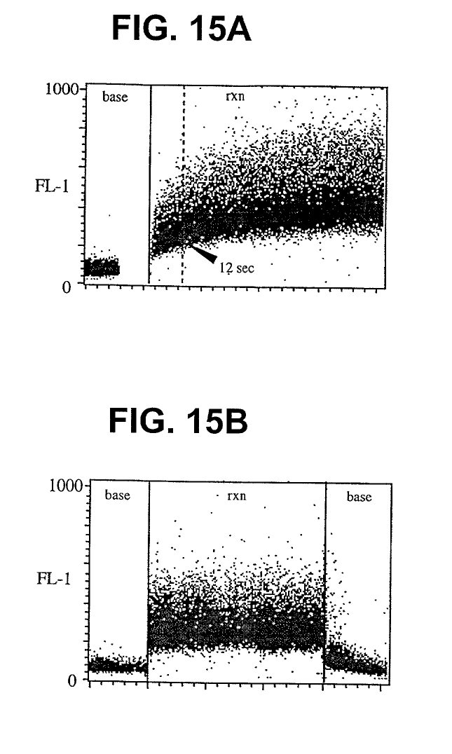

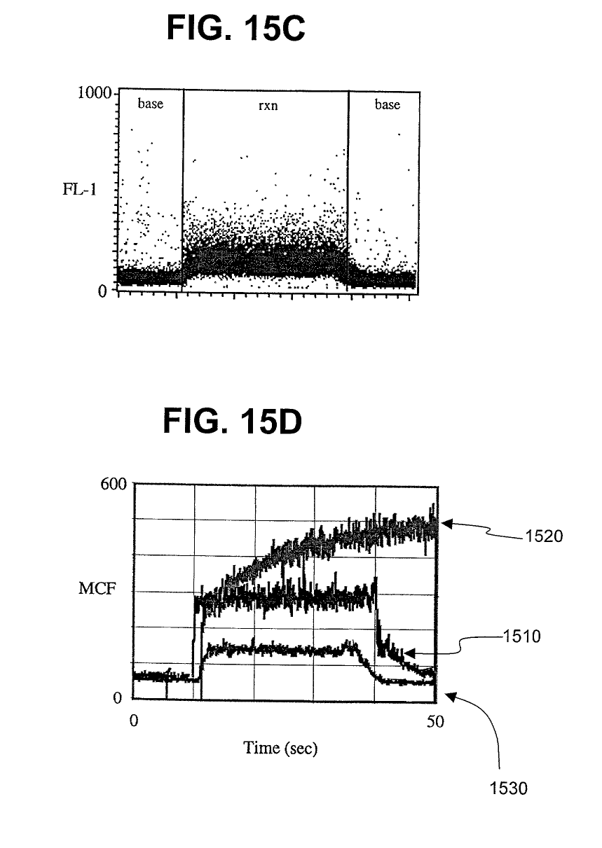

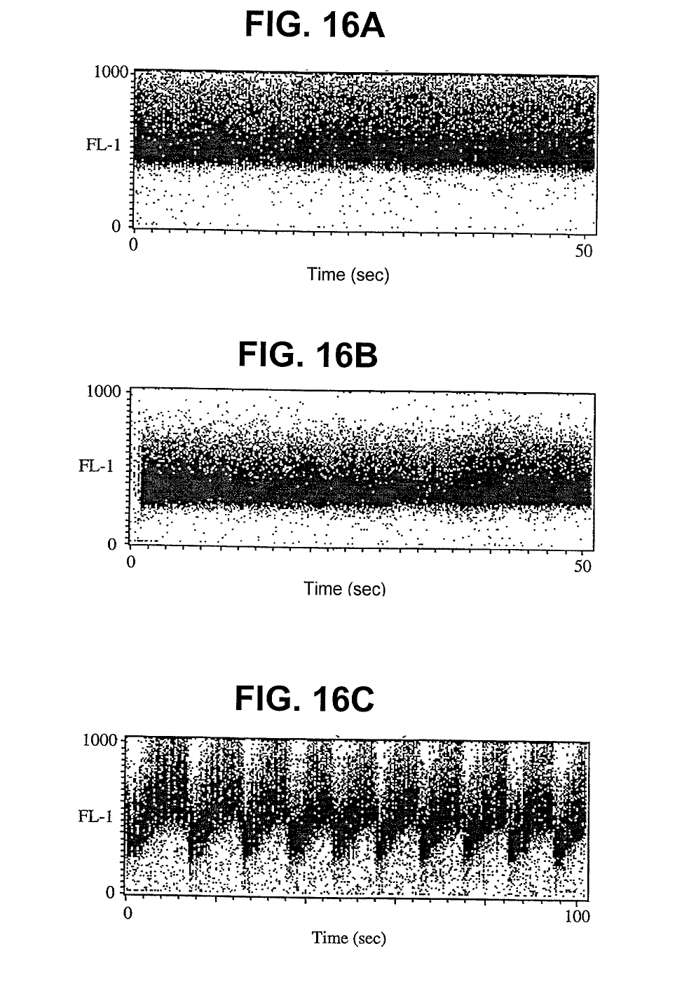

Experiments were done to determine the basis of the mixing. The results are shown in FIGS. 15A, 15B, 15C, 15D, 16A, 16B and 16C. The results shown in FIGS. 15A, 15B, 15C and 15D show the role of the peristaltic pump in the mixing. A comparison was done in the same series of experiments with the time course of the unquenching reaction, as shown in FIG. 15A, to the response of the beads delivered by peristaltic action, as shown in FIG. 15B, or syringe, as shown in FIG. 15C. The peristaltic action 1510 provided a response comparable to manual bulk phase mixing 1520 in the same time window whereas the syringes 1530 provided a smaller response consistent with the action of diffusive mixing alone in laminar flow (D).

As shown by FIGS. 16A, 16B and 16C, the experiments used an antibody to fluorescein to quench fluorescent target beads. If diffusion alone were at work, the expected results may be that a minimal quenching reaction would be observed. The present invention appreciates that antibody-containing wells led to quenching of the target beads with the magnitude anticipated. The experimental results showed that the quenching observed was equally effective with or without bubbles. There is a well-known mechanism that allows bubble-separated samples to mix called a driven cavity. In the present experiment, the recirculation streamlines of liquid, front to back, in a moving discrete drop, separated by bubbles, allows both convective mixing of solutes as well as molecular diffusion. Driven cavity microfluidic mixing allows samples that are in contact front to back to mix as the circulation lines travel from the front of the samples to the back. In contrast, laminar plugs separated by bubbles do not mix in the driven cavity microfluidic system because the circulation lines do not travel across the laminar plugs. The results of the experiment showing mixing in a system where diffusion is slow and bubbles are absent, may suggest a possibility of a different mechanism.

All of these results in total tend to suggest that the effective mixing observed in the microfluidic flow cytometry delivery system is neither due to diffusion nor to driven cavity mixing. Rather, all the results tend to suggest and may be consistent with pulsatile fluid motions, a phenomena which has been recognized, but which is not yet completely characterized.

FIGS. 15A, 15B, 15C and 15D show several comparisons of sample delivery by a peristaltic pump and a syringe for the unquenching reaction. FIG. 15A shows a FLI dot plot of fluorescence unquenching versus time for manual mixing of a 200 μl sample of beads combined with 200 μl aliquot of biotin (3.85×10⁻⁶ M final). FIG. 15B shows a FLI dot plots versus time for peristaltic delivery of 1:1 biotin and bead samples, as in FIG. 15A, flowing through the Y at 200 μl/min. The reaction time is 12 seconds based on flow through 35.5 cm of 254 μm ID tubing. The signal intensity produced by peristaltic action is similar to a homogeneously mixed solution at 12 seconds and is illustrated by the dotted line in FIG. 15. FIG. 15C shows a FLI dot plot versus time of biotin and beads, similar to the previous characteristics as above, using two syringes at a total of 200 μl/min flow rate. FIG. 15D is an overlay of mean channel fluorescence of data from FIGS. 15A, 15B and 15C. Manual bulk phase mixing 1520, peristaltic action 1510 and syringes 1530, which correspond, respectively to FIGS. 15A, 15B and 15C, are shown in FIG. 15D.

FIGS. 16A, 16B and 16C show several comparisons of mixing for a large molecule (antibody) quenching reaction in manual sample handling and peristaltic sample delivery with and without bubbles. The reaction of the antibody to fluorescein and unquenched FITC-biotin beads was examined after manual mixing and peristaltic mixing with or without bubbles. Data are plotted as time versus mean channel fluorescence. When unquenching is induced by adding biotin to streptavidin beads with FITC biotin as above, the quenching induced by the addition of 100 nM antibody to fluorescein occurs with a half-time 15 seconds (data not shown). FIG. 16A shows a peristaltic delivery of unquenched beads with bubbles. FIG. 16B shows a peristaltic delivery of unquenched beads from one side of the Y and 200 nM antibody from the other without bubbles. FIG. 16C shows that unquenched beads are allowed to interact with the antibody to fluorescein delivered from a multi-well plate. In delivery without bubbles, as in FIG. 16B, the beads are in laminar flow with the antibody, i.e. 200 nM. In delivery with bubbles, as in FIG. 16C, the antibody is contained in a well at 600 nM. The sample is diluted ˜⅓ during bubble formation, and spread unequally over several sample lengths, as shown in FIG. 14C, resulting in a peak concentration estimated to be no higher than 100 nM. Because of the slow diffusion of the antibody, the quenching observed in B and C appears to have resulted from mixing. The reaction time is 12 seconds.

Sample Carryover and Sample Throughput

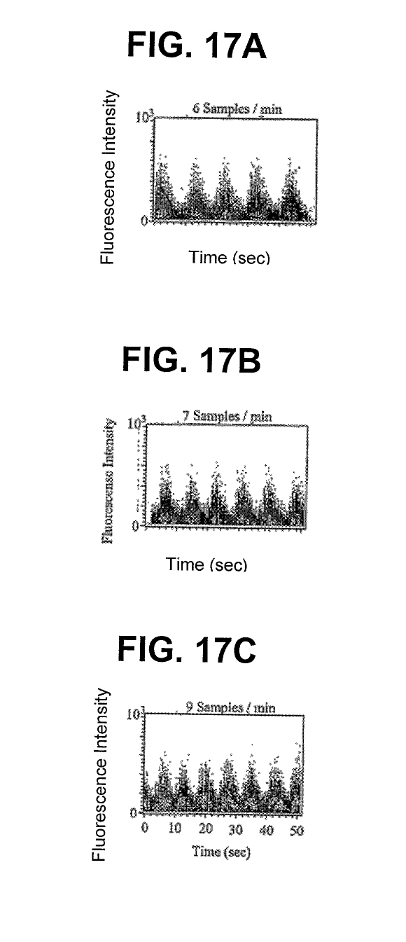

In the microfluidic mixing system of the present invention, it is important to reduce cross contamination or carryover of sequential samples. For the biotin samples, the degree of carryover is measured by the number of rinse steps necessary to recover the baseline fluorescence of beads whether quenching or unquenching. FIGS. 17A, 17B and 17C show that throughput can be increased by decreasing the number of rinse cycles as well as by reducing the number of wells sampled. Data are shown as dot plots versus time. The experiment was conducted as described in the above description of FIG. 13 except the number of buffer plugs was reduced by reducing the number of rinse wells from 9 to 8 resulting in throughput of 6 samples per minute as shown in FIG. 17A. Reducing the number of rinses to 7 from 8 results in a throughput of 7 samples per minute as shown in FIG. 17B. By dropping the number of rinses to 6 from 9 the throughput can be increased to 9 samples per minute as shown in FIG. 17C. In screening applications where positive compounds are likely to be few and far between in compound libraries, it is likely that the rate may be doubled again by halving the numbers of rinses. Thus, positive wells followed by negative wells will show a downward trend, while positive wells followed by positive will show a plateau.

Based on the above experiments, mixing occurs when samples introduced through a Y or T are separated by bubbles. This approach has not been used previously for high throughput or continuous screening applications. In microfluidic mixing systems of the present invention, the pulsatile action allows both convective mixing of solutes as well as molecular diffusion.

### EXAMPLE 4

Sample Mixing Using Pulsatile Fluid Motion

Another embodiment of the present invention, with respect to FIG. 17A-C, uses a Y junction and a peristaltic pump to drive the samples through the system. The sample flowing continuously is brought together in a Y with reagent samples from wells, which have been separated by bubbles. In the effluent stream, the particles and reagents are mixed, as a result of the peristaltic action, and the reagents from individual wells can be resolved. The sample volumes that may be mixed with this technology are submicroliter in volume and samples may be mixed at rates up to at least 100/samples per minute. With the geometry of the current invention, carry over between samples occurs within the Y that can be reduced if the mixing system is flushed with several volumes of buffer. The anticipated throughput for screening using this embodiment is expected to be at least 20 samples per minute. The high throughput approach and peristaltic mixing in the present embodiment serve to integrate autosamplers with submicroliter detection volumes for analysis in flow cytometry or in microfluidic channels.

With respect to FIGS. 14A-C, 15A-D and 17A-C, PI beads and Flow Check fluorescent beads were obtained from Beckman Coulter (Miami, Fla.). Biotin and fluorescein biotin (5-((N-(5-(N-(6-(biotinoyl)amino)hexanoyl)amino)pentyl)thioureidyl)fluorescein) were purchased from Molecular Probes (Eugene, Oreg.) and used without further purification. Antibody to fluorescein was prepared using standard methods.

6.2 μm diameter streptavidin-coated polystyrene beads (Spherotech Inc., Libertyville, Ill.) were obtained as 0.5% (w/v) suspensions according to the manufacturer's data sheet. A 200 μL volume suspension of streptavidin coated beads (3.7×10⁷ beads/mL) was diluted to 4 mL in TRIS buffer, (100 mM Tris HCl, 150 mM NaCl, 0.1% BSA, 0.02% sodium azide, pH 7.5). Subsequently, 1.0 μL of 8.7×10⁻⁶ M fluorescein biotin solution was added to the beads. The sample was continuously agitated for 20 minutes, then centrifuged and resuspended in 6.0 mL of buffer. The final bead suspension comprised of ˜1.1 million beads/mL with a million fluorescein biotin molecules per bead. For the unquenching reaction of the beads, biotin was added to the bead preparation. To follow a slower quenching reaction dependent upon diffusion of a higher molecular weight molecule, an antibody to fluorescein was used to quench the preparation of beads and biotin, similar to a previous report of quenching of fluoresceinated peptide bound to cell surface receptors.

With respect to FIGS. 11, 12A-B, 13, 14A-C, 15A-D, 16A-C and 17A-C, experiments were performed on a Becton Dickinson FacScan (San Jose, Calif.) equipped with a 488 Argon ion laser. Samples from a 96-well plate were delivered to the flow cytometer using a Gilson 215 autosampler (Middleton, Wis.). A Gilson interface module allowed for bi-directional communication between an MS DOS computer, a robotic probe arm and peristaltic pump. The probe was a 0.305 meter long (508 μm OD×254 μm ID) stainless steel tube (Small Parts Inc., Miami Lakes, Fla.). The probe was typically attached to 177 μm (00.75 in) ID flexible PVC tubing (Spectra Hardware Inc., Westmoreland City, Pa.) 1.5 meters in length and run through a peristaltic pump. This fluid line was attached to one leg of a ‘Y’-connector (#U-TCY-25 0.01 inch ID, Small Parts Inc.) The other leg of the Y-connector was coupled to similar tubing delivering a continuous stream of streptavidin-coated beads. Typically, the flow cytometer was connected via 1.5 m. tubing (254 μm, 0.01 in ID) attached to the third leg of the ‘Y’. All legs of the ‘Y’ fitting have an inner diameter of 254 um with a central triangular dead volume of 0.14 μL. A specialized fitting minimized sample disruption between the narrow ID delivery tubing and the flow cytometer intake tube, ID 0.016 inch (#U-4TX-25-12). In control experiments, the lengths of tubing were varied to test the effects of carryover as well as the time between the Y and the flow cytometer. In addition, automated syringes were used in place of the peristaltic pump to evaluate the contribution of pulsatile motion.

On-board software written in Microsoft Visual C⁺⁺ was used to control the speed and distance of probe motions in three dimensions. The speed of the peristaltic pump was manually controlled. Sample and bubble size were regulated by varying the time the probe was in a well or above a well intaking air. In a typical experiment, the peristaltic pump ran at 15 RPM. Sample plugs were removed from wells at sampling times of 400 ms per well. Sample-separating bubbles were generated during the time the probe was in transit between sample intakes (˜300 ms). The size of the sample plug was also regulated by the initial volume of sample in a given well. Biotin plugs were made smaller by keeping the initial volume of the biotin wells at 100 μL compared to the rinse wells, which contained 300 μL of buffer. For the sampling sequence comprising the repetitive delivery of a 3.85×10⁻⁶ M biotin plug and 9 rinses, the resultant stream of plugs consists of an estimated 0.6 μL of biotin and 0.9 μL of neat TRIS buffer. The biotin and buffer plugs subsequently combined with the continuous stream of fluorescein biotin-bearing beads at the “Y-junction. To track the onset of each biotin plug after mixing at the Y-junction Flow Check beads were included in each biotin sample well. The 96 well plate was periodically agitated to minimize settling of bead suspensions.

CELLQuest software (Becton-Dickinson) was used to acquire the time-resolved event clusters generated by rapid multi-well sampling. Event clusters representing the bead/biotin interactions were identified based on changes in fluorescence intensity and automatically analyzed via software algorithms. The algorithms calculate mean and median fluorescence intensity as well as event number and standard deviation of each event cluster. More detailed analysis such as washout sequences and sample carryover identification was done in Microsoft Excel via the off-line analysis of the data files using flow cytometry list-mode data files stored in FCS 2.0 format. It is worth noting that data acquisition occurs continuously which includes the air bubbles and fluid plugs simultaneously. The air bubbles between each fluid plug are denoted by the gaps in event clusters and signal discontinuity.

FIG. 15A shows the fluorescence intensity of unquenching versus time based for manual mixing of a 200 μl sample of beads combined with a 200 μl aliquot of biotin (7.7×10⁻⁶ M).

FIG. 15B shows fluorescence intensity dot plots versus time for peristaltic delivery of 1:1 biotin and bead samples using 200 μl sample of beads combined with a 200 μl aliquot of biotin (7.7×10⁻⁶ M). Here the beads and the biotin are flowing through the Y at 200 μl/min. The reaction time is 12 seconds based on flow through 35.5 cm of 254 μm ID tubing. The signal intensity produced by peristaltic action is similar to a homogenously mixed solution at 12 seconds and is illustrated by the dotted line in FIG. 15A.

FIG. 15C shows the results of an experiment conducted under the same conditions as in FIG. 15B. The fluorescence intensity dot plots versus time for mixing of biotin and beads is monitored. The beads and the biotin are delivered from two syringes through the Y at a total 200 μl/min flow rate.

FIG. 15D shows an overlay in mean channel fluorescence (MCF) of data from FIGS. 15A-C. The top MCF shown in FIG. 15D corresponds to data from FIG. 15A. The middle MCF corresponds to data from FIG. 15B. The bottom MCF corresponds to data from FIG. 15C.

The role of the peristaltic pump in enhancing mixing is shown by FIGS. 15A-C. In the same series of experiments, a comparison was made of the time course of the unquenching reaction shown in FIG. 15A to the response of the beads delivered by peristaltic action as shown in FIG. 15B or syringe as shown by FIG. 15C. The peristaltic action provided a response comparable to manual bulk phase mixing in the same time window whereas the syringes provide a smaller response consistent with the action of diffusive mixing alone as shown by FIG. 15D.

Although the present invention has been fully described in conjunction with the preferred embodiment thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A microfluidic mixing apparatus comprising:
first driving means for driving a plurality of reagent samples from a plurality of respective source wells into a first fluid flow stream;
second driving means for introducing a separation gas between each of said plurality of reagent samples in said first fluid flow stream;
means for driving a second fluid flow stream comprising a plurality of particles;
a junction device comprising:
a first inlet port for receiving said first fluid flow stream;
a second inlet port for receiving said second fluid flow stream;
a first reaction zone for forcing mixing between said first fluid flow stream and said second fluid flow stream to thereby form a reaction product stream; and
an outlet port for allowing said reaction product stream to exit said junction device;
a second reaction zone where said plurality of reagent samples and said plurality of particles mix to form a plurality of reaction products, said reaction zone communicating with said outlet port;
reaction product driving means for driving said reaction product stream through said reaction zone; and
means for selectively analyzing said reaction product stream for said reaction products.

2. The microfluidic mixing apparatus of claim 1, wherein said first driving means comprises an autosampler.

3. The microfluidic mixing apparatus of claim 2, wherein said autosampler includes a probe and said microfluidic mixing apparatus includes a means for exposing a probe tip of said probe to a jet of gas to remove liquid from said probe tip.

4. The microfluidic mixing apparatus of claim 2, wherein said autosampler includes a probe having a conical tip.

5. The microfluidic mixing apparatus of claim 2, wherein said autosampler includes a hydrophobic probe.

6. The microfluidic mixing apparatus of claim 5, wherein said probe comprises a hydrophobic material.

7. The microfluidic mixing apparatus of claim 5, wherein said probe is coated with a hydrophobic material.

8. The microfluidic mixing apparatus of claim 2, wherein said first driving means further comprises a first fluid flow stream peristaltic pump.

9. The microfluidic mixing apparatus of claim 8, wherein a portion of said fluid flow stream passing through said first fluid flow stream peristaltic pump is contained within a high speed multi-sample tube.

10. The microfluidic mixing apparatus of claim 8, wherein said first fluid flow stream peristaltic pump is located along said fluid flow stream between said autosampler and said junction device.

11. The microfluidic mixing apparatus of claim 8, wherein said second driving means comprises a second fluid flow stream peristaltic pump.

12. The microfluidic mixing apparatus of claim 11, wherein a portion of said second fluid flow stream passing through said second fluid flow stream peristaltic pump is contained within a high speed multi-sample tube.

13. The microfluidic mixing apparatus of claim 11, wherein said first fluid flow stream peristaltic pump and said second fluid flow stream peristaltic pump comprise the same peristaltic pump.

14. The microfluidic mixing apparatus of claim 1, wherein said reaction product driving means comprises said first driving means and said second driving means.

15. The microfluidic mixing apparatus of claim 14, wherein said first driving means, said second driving means and said reaction product driving means comprises the same peristaltic pump.

16. The microfluidic mixing apparatus of claim 1, further comprising a first tubing for containing said first fluid flow stream, a second tubing for containing said second fluid flow stream and a reaction product tubing for containing said reaction product stream.

17. The microfluidic mixing apparatus of claim 16, wherein said microfluidic mixing apparatus includes a unibody flow apparatus comprising said first tubing, said second tubing, said reaction product tubing, and said junction device.

18. The microfluidic mixing apparatus of claim 16, wherein said first tubing comprises high speed multi-sample tubing.

19. The microfluidic mixing apparatus of claim 18, wherein said high speed multi-sample tubing comprises PVC tubing having an inner diameter about 0.005 to about 0.02 inches and a wall thickness of about 0.01 to about 0.03 inches.

20. The microfluidic mixing apparatus of claim 18, wherein said high speed multi-sample tubing comprises PVC tubing having an inner diameter about 0.01 inches and a wall thickness of about 0.01 to about 0.03 inches.

21. The microfluidic mixing apparatus of claim 16, wherein said second tubing comprises high speed multi-sample tubing.

22. The microfluidic mixing apparatus of claim 21, wherein said high speed multi-sample tubing comprises PVC tubing having an inner diameter about 0.005 to about 0.02 inches and a wall thickness of about 0.01 to about 0.03 inches.

23. The microfluidic mixing apparatus of claim 21, wherein said high speed multi-sample tubing comprises PVC tubing having an inner diameter about 0.01 inches and a wall thickness of about 0.01 to about 0.03 inches.

24. The microfluidic mixing apparatus of claim 16, wherein said reaction product tubing comprises high-speed multi-sample tubing.

25. The microfluidic mixing apparatus of claim 24, wherein said high speed multi-sample tubing comprises PVC tubing having an inner diameter about 0.005 to about 0.02 inches and a wall thickness of about 0.01 to about 0.03 inches.

26. The microfluidic mixing apparatus of claim 24, wherein said high speed multi-sample tubing comprises PVC tubing having an inner diameter about 0.01 inches and a wall thickness of about 0.01 to about 0.03 inches.

27. The microfluidic mixing apparatus of claim 1, wherein said first inlet port, said second inlet port and said outlet port each have an inner diameter about 0.005 to about 0.02 inches.

28. The microfluidic mixing apparatus of claim 1, wherein said first inlet port, said second inlet port and said outlet port each have an inner diameter about 0.01 inches.

29. The micro fluidic mixing apparatus of claim 1, wherein said separation gas comprises air.

30. The microfluidic mixing apparatus of claim 1, wherein said plurality of reagent samples are homogenous.

31. The microfluidic mixing apparatus of claim 1, wherein said plurality of reagent samples are heterogeneous.

32. The microfluidic mixing apparatus of claim 1, wherein said particles comprise biomaterials.

33. The microfluidic mixing apparatus of claim 32, wherein said biomaterials are fluorescently tagged.

34. The microfluidic mixing apparatus of claim 1, further comprising a well plate including said plurality of respective source wells.

35. The microfluidic mixing apparatus of claim 34, wherein said well plate includes at least 60 source wells.

36. The microfluidic mixing apparatus of claim 34, wherein said well plate includes at least 72 source wells.

37. The microfluidic mixing apparatus of claim 34, wherein said well plate includes at least 96 source wells.

38. The microfluidic mixing apparatus of claim 34, wherein said well plate includes at least 384 source wells.

39. The microfluidic mixing apparatus of claim 34, wherein said well plate includes at least 1536 source wells.

40. The microfluidic mixing apparatus of claim 34, wherein said well plate includes wells having a conical shape.

41. The microfluidic mixing apparatus of claim 34, wherein said well plate is mounted in an inverted position.

42. The microfluidic mixing apparatus of claim 1, further comprising a means for injecting a buffer fluid between adjacent reagent samples in said first fluid flow stream.

43. The microfluidic mixing apparatus of claim 1, wherein at least one of said plurality of reagent samples comprises a drug.

44. The microfluidic mixing apparatus of claim 1, wherein said junction device is Y-shaped.

45. The microfluidic mixing apparatus of claim 44, wherein the angle between any two of said first inlet port, said second inlet port and said outlet port is 120°.

46. The microfluidic mixing apparatus of claim 1, wherein said junction device is T-shaped.

47. The microfluidic mixing apparatus of claim 1, further comprising a first inlet tube connected to said first inlet port, a second inlet tube connected to said second inlet port and an outlet tube connected to said outlet port, wherein said first inlet tube and said first inlet port have the same inner diameter, wherein said second inlet tube and said second inlet port have the same inner diameter, and said outlet tube and said outlet port have the same inner diameter.

48. The microfluidic mixing apparatus of claim 47, wherein said first inlet port, said second inlet port and said outlet port each have the same interior diameter.

49. The microfluidic mixing apparatus of claim 47, wherein said first inlet port and said second inlet port have the same inner diameter and said outlet port has a different inner diameter from said first inlet port and said second inlet port.

50. The microfluidic mixing apparatus of claim 49, wherein said outlet port has a larger inner diameter than said first inlet port and said second inlet port.

51. A method for mixing materials comprising:
driving a first fluid flow stream comprising a plurality of reagent samples separated by gas bubbles through a second inlet port of a junction device;
driving a second fluid flow stream comprising particles through a first inlet port of said junction device;
mixing said first fluid flow stream and said second fluid flow stream in a reaction zone in said junction device to form a reaction product stream; and
driving said reaction product stream through an outlet port of said junction device.

52. The method of claim 51, wherein said gas comprises air.

53. The method of claim 51, wherein said junction device comprises a Y-shaped junction device.

54. The method of claim 53, wherein the angle between any two of the group consisting of said first inlet port, said second inlet port and said outlet port is 120°.

55. The method of claim 51, wherein said junction devices a T-shaped junction device and said outlet port is perpendicular to said first entry port and said second entry port.

56. The method of claim 51, wherein said first inlet port has a diameter of 0.005 to 0.02 inches.

57. The method of claim 51, wherein said first inlet port has a diameter of 0.01 inches.

58. The method of claim 51, wherein said second inlet port has a diameter of 0.005 to 0.02 inches.

59. The method of claim 51, wherein said second inlet port has a diameter of 0.01 inches.

60. The method of claim 51, wherein said outlet port has a diameter of 0.005 to 0.02 inches.

61. The method of claim 51, wherein said outlet port has a diameter of 0.01 inches.

62. The method of claim 51, further comprising reacting said particles with each of said plurality of reagent samples of said reaction product stream in said reaction zone to thereby form a plurality of reaction product samples.

63. The method of claim 62, further selectively analyzing each of said plurality of reaction product samples after said plurality of reaction product samples have passed through said reaction zone.

64. The method of claim 63, by which said reaction product samples are sorted on a particle by particle basis in a flow cytometer.

65. The method of claim 51, further comprising intaking said plurality of reagent samples into said first fluid flow stream from a plurality of respective wells.

66. The method of claim 51, wherein said plurality of reagent samples are separated from each other in said first fluid flow stream by intaking air into said fluid flow stream between intaking adjacent samples of said plurality of samples.

67. The method of claim 51, wherein at least 6 reaction product samples are selectively analyzed per minute.

68. The method of claim 51, wherein at least 60 reaction product samples are selectively analyzed per minute.

69. The method of claim 51, wherein at least 120 reaction product samples are selectively analyzed per minute.

70. The method of claim 51, wherein at least 240 reaction product samples are selectively analyzed per minute.

71. The method of claim 51, wherein said plurality of reagent samples are homogenous.

72. The method of claim 51, wherein said plurality of reagent samples are heterogeneous.

73. The method of claim 51, wherein said particles comprise biomaterials.

74. The method of claim 73, wherein said biomaterials are fluorescently tagged.

75. The method of claim 51, wherein each of said reagent samples has a reagent sample size ranging from about 0.1 to about 10 μl.

76. The method of claim 51, wherein said reagent sample product flows in said reagent sample product fluid flow stream at a flow rate of about 0.1 to about 10 μl/sec.

77. The method of claim 51, further comprising injecting a buffer fluid between at least two adjacent reagent samples in said first fluid flow stream

78. The method of claim 51, wherein said plurality of reagent samples comprises at least one drug.
